(19) United States
(12) Patent Application Publication
(10) Pub. No.: US 2005/0049204 A1
(43) Pub. Date: Mar. 3, 2005

(54) Compounds for the treatment of flaviviridae infections

(76) Inventors: Michael J. Otto, Lilburn, GA (US); Lieven Stuyver, Herzele (BE)

Correspondence Address: KING & SPALDING LLP, 191 PEACHTREE STREET, N.E., ATLANTA, GA, 30303-1763, US

(21) Appl. No.: 10/812,448

(22) Filed: Mar. 29, 2004

Related U.S. Application Data
(60) Provisional application No. 60/458,635, filed on Mar. 28, 2003.

(51) Int. Cl. A61K 31/7056; A61K 31/675; A61K 31/425

(52) U.S. Cl. 514/23; 514/89; 514/372

(57) ABSTRACT

The disclosed invention is a composition for and a method of treating a Flaviviridae infections, such as bovine viral diarrhea virus (“BVDV”), Dengue Virus (DENV), West Nile Virus (WNV) and hepatitis C virus (HCV), as well as abnormal cellular proliferation, in a host, including animals, and especially humans, using a nucleoside of general formula (I)-(V) or N-(phosphonoacetyl)-L-aspartate (PALA), or a pharmaceutically acceptable salt or prodrug thereof.

### CROSS-REFERENCE TO RELATED APPLICATIONS

This application claims priority to U.S. Provisional Application No. 60/458,635, filed Mar. 28, 2003.

### FIELD OF THE INVENTION

The present invention includes compounds and methods for the treatment of Flaviviridae infections, such as bovine viral diarrhea virus (“BVDV”), Dengue Virus (DENV), West Nile Virus (WNV) and hepatitis C virus (HCV), as well as abnormal cellular proliferation.

### BACKGROUND OF THE INVENTION

Flaviviridae

The Flaviviridae family of viruses comprises at least three distinct genera: pestiviruses, which cause disease in cattle and pigs; flaviviruses, which are the primary cause of diseases such as dengue fever and yellow fever; and hepaciviruses, whose sole member is HCV. The flavivirus genus includes more than 68 members separated into groups on the basis of serological relatedness (Calisher et al., J. Gen. Virol, 1993, 70, 37-43). Clinical symptoms vary and include fever, encephalitis and hemorrhagic fever (Fields Virology, Editors: Fields, B. N., Knipe, D. M., and Howley, P. M., Lippincott-Raven Publishers, Philadelphia, Pa., 1996, Chapter 31, 931-959). Flaviviruses of global concern that are associated with human disease include the dengue hemorrhagic fever viruses (DHF), yellow fever virus, West Nile virus, shock syndrome and Japanese encephalitis virus (Halstead, S. B., Rev. Infect. Dis., 1984, 6, 251-264; Halstead, S. B., Science, 239:476-481, 1988; Monath, T. P., New Eng. J. Med., 1988, 319, 641-643).

The pestivirus genus includes bovine viral diarrhea virus (BVDV), classical swine fever virus (CSFV, also called hog cholera virus) and border disease virus (BDV) of sheep (Moennig, V. et al. Adv. Vir. Res. 1992, 41, 53-98). Pestivirus infections of domesticated livestock (cattle, pigs and sheep) cause significant economic losses worldwide. BVDV causes mucosal disease in cattle and is of significant economic importance to the livestock industry (Meyers, G. and Thiel, H.-J., Advances in Virus Research, 1996, 47, 53-118; Moennig V., et al, Adv. Vir. Res. 1992, 41, 53-98). Human pestiviruses have not been as extensively characterized as the animal pestiviruses. However, serological surveys indicate considerable pestivirus exposure in humans.

Pestiviruses and hepaciviruses are closely related virus groups within the Flaviviridae family. Other closely related viruses in this family include the GB virus A, GB virus A-like agents, GB virus-B and GB virus-C (also called hepatitis G virus, HGV). The hepacivirus group (hepatitis C virus; HCV) consists of a number of closely related but genotypically distinguishable viruses that infect humans. There are approximately 6 HCV genotypes and more than 50 subtypes. HCV is a major cause of hepatitis globally. Most HCV infections become persistent and about 75% of cases develop chronic liver disease. Chronic HCV infection can lead to development of cirrhosis, hepatocellular carcinoma and liver failure. Due to the similarities between pestiviruses and hepaciviruses, combined with the poor ability of hepaciviruses to grow efficiently in cell culture, bovine viral diarrhea virus (BVDV) is often used as a surrogate to study the HCV virus.

The genetic organization of pestiviruses and hepaciviruses is very similar. These positive stranded RNA viruses possess a single large open reading frame (ORF) encoding all the viral proteins necessary for virus replication. These proteins are expressed as a polyprotein that is co- and post-translationally processed by both cellular and virus-encoded proteinases to yield the mature viral proteins. The viral proteins responsible for the replication of the viral genome RNA are located within approximately the carboxy-terminal two-thirds of the ORF and are termed nonstructural (NS) proteins. The genetic organization and polyprotein processing of the nonstructural protein portion of the ORF for pestiviruses and hepaciviruses is very similar. For both the pestiviruses and hepaciviruses, the mature nonstructural (NS) proteins, in sequential order from the amino-terminus of the nonstructural protein coding region to the carboxy-terminus of the ORF, consist of p7, NS2, NS3, NS4A, NS4B, NS5A, and NS5B.

The NS proteins of pestiviruses and hepaciviruses share sequence domains that are characteristic of specific protein functions. For example, the NS3 proteins of viruses in both groups possess amino acid sequence motifs characteristic of serine proteinases and of helicases (Gorbalenya et al. (1988) Nature 333:22; Bazan and Fletterick (1989) Virology 171:637-639; Gorbalenya et al. (1989) Nucleic Acid Res. 17.3889-3897). Similarly, the NS5B proteins of pestiviruses and hepaciviruses have the motifs characteristic of RNA-directed RNA polymerases (Koonin, E. V. and Dolja, V. V. (1993) Crit. Rev. Biochem. Molec. Biol. 28:375-430).

Furthermore, the actual roles and functions of the NS proteins of pestiviruses and hepaciviruses in the lifecycle of the viruses are directly analogous. In both cases, the NS3 serine proteinase is responsible for all proteolytic processing of polyprotein precursors downstream of its position in the ORF (Wiskerchen and Collett (1991) Virology 184:341-350; Bartenschlager et al. (1993) J. Virol. 67:3835-3844; Eckart et al. (1993) Biochem. Biophys. Res. Comm. 192:399-406; Grakoui et al. (1993) J. Virol. 67:2832-2843; Grakoui et al. (1993) Proc. Natl. Acad. Sci. USA 90:10583-10587; Hijikata et al. (1993) J. Virol. 67:4665-4675; Tome et al. (1993) J. Virol. 67:4017-4026). The NS4A protein, in both cases, acts as a cofactor with the NS3 serine protease (Bartenschlager et al. (1994) J. Virol. 68:5045-5055; Failla et al. (1994) J. Virol. 68: 3753-3760; Lin et al. (1994) 68:8147-8157; Xu et al. (1997) J. Virol. 71:5312-5322). The NS3 protein of both viruses also functions as a helicase (Kim et al. (1995) Biochem. Biophys. Res. Comm. 215: 160-166; Jin and Peterson (1995) Arch. Biochem. Biophys. 323:47-53; Warrener and Collett (1995) J. Virol. 69:1720-1726). Finally, the NS5B proteins of pestiviruses and hepaciviruses have the predicted RNA-directed RNA polymerases activity (Behrens et al. (1996) EMBO J. 15:12-22; Lachmannet al. (1997) J. Virol. 71:8416-8428; Yuan et al. (1997) Biochem. Biophys. Res. Comm. 232:231-235; Hagedorn, PCT WO 97/12033; Zhong et al. (1998) J. Virol. 72.9365-9369).

Interferons (IFNs) are compounds that have been commercially available for the treatment of chronic hepatitis for nearly a decade. IFNs are glycoproteins produced by immune cells in response to viral infection. IFNs inhibit viral replication of many viruses, including HCV, and when used as the sole treatment for hepatitis C infection, IFN suppresses serum HCV-RNA to undetectable levels. Additionally, IFN normalizes serum amino transferase levels. Unfortunately, the effects of IFN are temporary and a sustained response occurs in only 8%-9% of patients chronically infected with HCV (Gary L. Davis. Gastroenterology 118:S104-S114, 2000).

A number of patents disclose HCV treatments using interferon-based therapies. For example, U.S. Pat. No. 5,980,884 to Blatt et al. discloses methods for retreatment of patients afflicted with HCV using consensus interferon. U.S. Pat. No. 5,942,223 to Bazer et al. discloses an anti-HCV therapy using ovine or bovine interferon-tau. U.S. Pat. No. 5,928,636 to Alber et al. discloses the combination therapy of interleukin-12 and interferon alpha for the treatment of infectious diseases including HCV. U.S. Pat. No. 5,908,621 to Glue et al. discloses the use of polyethylene glycol modified interferon for the treatment of HCV. U.S. Pat. No. 5,849,696 to Chretien et al. discloses the use of thymosins, alone or in combination with interferon, for treating HCV. U.S. Pat. No. 5,830,455 to Valtuena et al. discloses a combination HCV therapy employing interferon and a free radical scavenger. U.S. Pat. No. 5,738,845 to Imakawa discloses the use of human interferon tau proteins for treating HCV. Other interferon-based treatments for HCV are disclosed in U.S. Pat. No. 5,676,942 to Testa et al., U.S. Pat. No. 5,372,808 to Blatt et al., and U.S. Pat. No. 5,849,696.

Ribavirin (1-β-D-ribofuranosyl-1-1,2,4-triazole-3-carboxamide) is a synthetic, non-interferon-inducing, broad spectrum antiviral nucleoside analog. It is sold under the trade names Virazole™ (The Merck Index, 11th edition, Editor: Budavari, S., Merck & Co., Inc., Rahway, N.J., p1304, 1989); Rebetol (Schering Plough) and Co-Pegasus (Roche). U.S. Pat. No. 3,798,209 and RE29,835 (ICN Pharmaceuticals) disclose and claim ribavirin. Ribavirin is structurally similar to guanosine, and has in vitro activity against several DNA and RNA viruses including Flaviviridae (Gary L. Davis. Gastroenterology 118:S104-S114, 2000). U.S. Pat. No. 4,211,771 (to ICN Pharmaceuticals) discloses the use of ribavirin as an antiviral agent. Ribavirin reduces serum amino transferase levels to normal in 40% of patients, but it does not lower serum levels of HCV-RNA (Gary L. Davis. Gastroenterology 118:S104-S114, 2000). Thus, ribavirin alone is not effective in reducing viral RNA levels. Additionally, ribavirin has significant toxicity and is known to induce anemia.

Schering-Plough sells ribavirin as Rebetol® capsules (200 mg) for administration to patients with HCV. The U.S. FDA has approved Rebetol capsules to treat chronic HCV infection in combination with Schering's alpha interferon-2b products Intron® A and PEG-Intron™. Rebetol capsules are not approved for monotherapy (i.e., administration independent of Intron®A or PEG-Intron), although Intron A and PEG-Intron are approved for monotherapy (i.e., administration without ribavirin). Hoffman La Roche is selling ribavirin under the name Co-Pegasus in Europe and the United States, also for use in combination with interferon for the treatment of HCV. Other alpha interferon products include Roferon-A (Hoffmann-La Roche), Infergen® (Intermune, formerly Amgen's product), and Wellferon® (Wellcome Foundation) are currently FDA-approved for HCV monotherapy. Interferon products currently in development for HCV include: Roferon-A (interferon alpha-2a) by Roche, PEGASYS (pegylated interferon alpha-2a) by Roche, INFERGEN (interferon alfacon-1) by InterMune, OMNIFERON (natural interferon) by Viragen, ALBUFERON by Human Genome Sciences, REBIF (interferon beta-1a) by Ares-Serono, Omega Interferon by BioMedicine, Oral Interferon Alpha by Amarillo Biosciences, and Interferon gamma-1b by InterMune.

The combination of IFN and ribavirin for the treatment of HCV infection has been reported to be effective in the treatment of IFN naïve patients (Battaglia, A. M. et al., Ann. Pharmacother. 34:487-494, 2000). Combination treatment is effective both before hepatitis develops and when histological disease is present (Berenguer, M. et al. Antivir. Ther. 3(Suppl. 3):125-136, 1998). Currently, the most effective therapy for HCV is combination therapy of pegylated interferon with ribavirin (2002 NIH Consensus Development Conference on the Management of Hepatitis C). However, the side effects of combination therapy can be significant and include hemolysis, flu-like symptoms, anemia, and fatigue (Gary L. Davis. Gastroenterology 118:S104-S114, 2000).

Other compounds currently in clinical development for treatment of hepatitis c virus include: Interleukin-10 by Schering-Plough, IP-501 by Interneuron, Merimebodib VX-497 by Vertex, AMANTADINE (Symmetrel) by Endo Labs Solvay, HEPTAZYME by RPI, IDN-6556 by Idun Pharma., XTL-002 by XTL., HCV/MF59 by Chiron, CIVACIR by NABI, LEVOVIRIN by ICN, VIRAMIDINE by ICN, ZADAXIN (thymosin alfa-1) by Sci Clone, CEPLENE (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

Idenix Pharmaceuticals, Ltd. discloses branched nucleosides, and their use in the treatment of HCV and flaviviruses and pestiviruses in US Patent Publication No. 2003/0050229 A1 and US Patent Publication No. 2003/0060400 A1, which correspond to International Publication Nos. WO 01/90121 and WO 01/92282. A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier. See also WO 03/026589 and WO 03/026675. Idenix Pharmaceuticals, Ltd. Also discloses pharmaceutically acceptable branched nucleoside prodrugs, and their use in the treatment of HCV and flaviviruses and pestiviruses in prodrugs. See PCT Publication Nos. WO 04/002422, WO 04/002999, and WO 04/003000.

Emory University and the University of Georgia Research Foundation, Inc. (UGARF) discloses the use of 2′-fluoronucleosides for the treatment of HCV in U.S. Pat. No. 6,348,587. See also International Patent Publication WO 99/43691.

BioChem Pharma Inc. (now Shire Biochem, Inc.) disclosed the use of various 1,3-dioxolane nucleosides for the treatment of a Flaviviridae infection in International Publication No. WO 01/32153 (PCT/CA00/01316; filed Nov. 3, 2000).

BioChem Pharma Inc. (now Shire Biochem, Inc.) also disclosed various other 2′-halo, 2′-hydroxy and 2′-alkoxy nucleosides for the treatment of a Flaviviridae infection in International Publication No. WO 01/60315 (PCT/CA01/00197; filed Feb. 19, 2001).

ICN Pharmaceuticals, Inc. discloses various nucleoside analogs that are useful in modulating immune response in U.S. Pat. Nos. 6,495,677 and 6,573,248. See also WO 98/16184, WO 01/68663, and WO 02/03997.

US Patent Publication Nos. 2003/083307 A1 and US 2003/008841 A1, and the corresponding International Patent Publication Nos. WO 02/18404 (PCT/EP01/09633; published Aug. 21, 2001); WO 02/100415 and WO 02/094289, filed by F. Hoffmann-La Roche AG discloses various nucleoside analogs for the treatment of HCV RNA replication.

Pharmasset Limited discloses various nucleosides and antimetabolites for the treatment of a variety of viruses, including Flaviviridae, and in particular HCV, in WO 02/32920, WO 01/79246, WO 02/48165, WO 03/068162, WO 03/068164 and 2004/013298.

Merck & Co., Inc. and Isis Pharmaceuticals disclose in US Patent Publication No. 2002/0147160 and the corresponding International Patent Publication Nos. WO 02/057425 (PCT/US02/01531; filed Jan. 18, 2002) and WO 02/057287 (PCT/US02/03086; filed Jan. 18, 2002) various nucleosides, and in particular several pyrrolopyrimidine nucleosides, for the treatment of viruses whose replication is dependent upon RNA-dependent RNA polymerase, including Flaviviridae, and in particular HCV. See also WO 2004/003138, WO 2004/007512, and WO 2004/009020.

US Patent Publication No. 2003/028013 A1 as well as International Patent Publication Nos. WO 03/051899, WO 03/061576, WO 03/062255 WO 03/062256, WO 03/062257, and WO 03/061385, filed by Ribapharm, also are directed to the use of certain nucleoside analogs to treat hepatitis C virus.

Abnormal Cellular Proliferation

Cellular differentiation, growth, function and death are regulated by a complex network of mechanisms at the molecular level in a multicellular organism. In the healthy animal or human, these mechanisms allow the cell to carry out its designed function and then die at a programmed rate.

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. In normal skin the time required for a cell to move from the basal layer to the upper granular layer is about five weeks. In psoriasis, this time is only 6 to 9 days, partially due to an increase in the number of proliferating cells and an increase in the proportion of cells which are dividing (G. Grove, Int. J. Dermatol. 18:111, 1979). Approximately 2% of the population in the United States has psoriasis, occurring in about 3% of Caucasian Americans, in about 1% of African Americans, and rarely in Native Americans. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. The advanced lesions of atherosclerosis result from an excessive inflammatory-proliferative response to an insult to the endothelium and smooth muscle of the artery wall (Ross, R. Nature, 1993, 362:801-809). Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells (See, e.g., Harris, E. D., Jr., The New England Journal of Medicine, 1990, 322: 1277-1289), and to be caused by autoantibodies produced against collagen and IgE.

Other disorders that can include an abnormal cellular proliferative component include Behcet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

A tumor, also called a neoplasm, is a new growth of tissue in which the multiplication of cells is uncontrolled and progressive. A benign tumor is one that lacks the properties of invasion and metastasis and is usually surrounded by a fibrous capsule. A malignant tumor (i.e., cancer) is one that is capable of both invasion and metastasis. Malignant tumors also show a greater degree of anaplasia (i.e., loss of differentiation of cells and of their orientation to one another and to their axial framework) than benign tumors.

Approximately 1.2 million Americans are diagnosed with cancer each year, 8,000 of which are children. In addition, 500,000 Americans die from cancer each year in the United States alone. Prostate and lung cancers are the leading causes of death in men while breast and lung cancer are the leading causes of death in women. It is estimated that cancer-related costs account for about 10% of the total amount spent on disease treatment in the United States (see CNN.Cancer.Facts: http://www.cnn.com/HEALTH/9511/conquer_cancer/facts/index.html, page 2 of 2, Jul. 18, 1999).

Proliferative disorders are currently treated by a variety of classes of compounds including alkylating agents, antimetabolites, natural products, enzymes, biological response modifiers, miscellaneous agents, radiopharmaceuticals (for example, Y-90 tagged to hormones or antibodies), hormones and antagonists.

Toxicity associated with therapy for abnormally proliferating cells, including cancer, is due in part to a lack of selectivity of the drug for diseased versus normal cells. To overcome this limitation, therapeutic strategies that increase the specificity and thus reduce the toxicity of drugs for the treatment of proliferative disorders are being explored. One such strategy that is being aggressively pursued is drug targeting.

In view of the severity of these diseases associated with a Flaviviridae infection and/or abnormally proliferating cells, including cancer, and their pervasiveness in animals, including humans, there is a need to provide a compound, method and composition for the treatment of a host, including animals and especially humans, infected with a Flaviviridae, including flaviviruses, pestiviruses, or hepaciviruses, such as HCV, and/or abnormally proliferating cells.

It is a particular object of the present invention to provide a compound, method and composition for the treatment of a host, including animals and especially humans, infected with a Flaviviridae virus.

It is a further object to provide a compound, method and composition for the treatment of a host, including animals and especially humans, infected with hepatitis C virus (HCV).

It is another object of the present invention to provide a compound, method and composition for the treatment of a host, including animals and especially humans, with abnormal cellular proliferation.

It is yet another object to provide a compound, method and composition for the treatment of a host, including animals and especially humans, with a malignant tumor.

### SUMMARY OF THE INVENTION

The present invention provides a β-D or β-L nucleoside of formula (I)-(V) or its pharmaceutically acceptable salt and/or prodrug, including an ester, for the treatment of a host infected with a Flaviviridae, including flaviviruses, pestiviruses, or hepaciviruses, such as HCV. Alternatively, the β-D or β-L nucleoside (I)-(V) or its pharmaceutically acceptable salt and/or prodrug, including an ester, can be used for the treatment of abnormal cellular proliferation.

The present invention also provides an anti-viral or anti-proliferative effective agent, N-(phosphonoacetyl)-L-aspartate (PALA), or its pharmaceutically acceptable salt and/or prodrug, for the treatment of a host infected with a Flaviviridae, including flaviviruses, pestiviruses, or hepaciviruses, such as HCV. Alternatively, PALA, or its pharmaceutically acceptable salt or prodrug, can be used for the treatment of abnormal cellular proliferation.

Specifically, the invention also includes methods for treating or preventing the following:
(a) a Flaviviridae infection, including all members of the Hepacivirus genus (HCV), Pestivirus genus (BVDV, CSFV, BDV), or Flavivirus genus (Dengue virus, Japanese encephalitis virus group (including West Nile Virus), and Yellow Fever virus); and/or
(b) abnormal cellular proliferation, including malignant tumors.

In one embodiment of the invention, the anti-viral or anti-proliferative effective nucleoside is a carbocyclic nucleoside of the general formula (I) to (II): 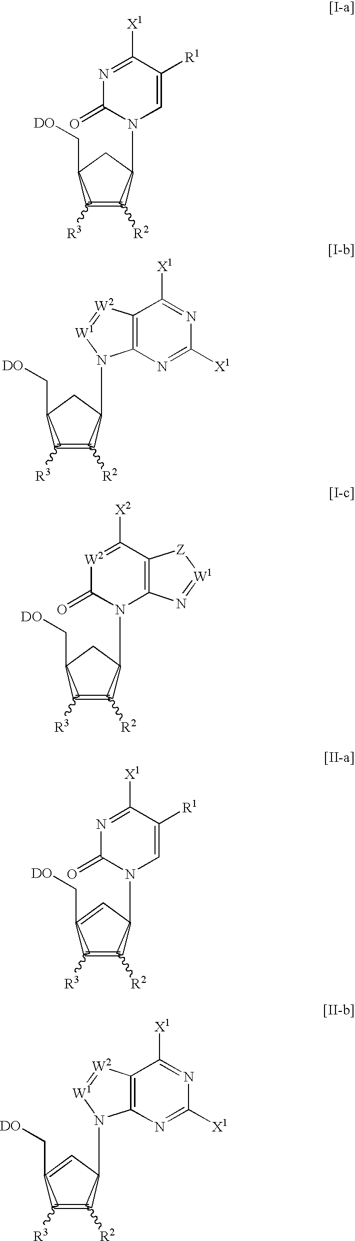 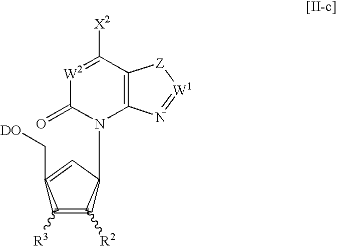
or a pharmaceutically acceptable salt and/or prodrug, including an ester, thereof, wherein:
each D is hydrogen, alkyl, acyl, monophosphate, diphosphate, triphosphate, monophosphate ester, diphosphate ester, triphosphate ester, phospholipid or amino acid;
each W¹ and W² is independently N, CH, CX² or CR¹;
each X¹ is independently NH₂, NHR⁴, NR⁴R^{4′}, NHOR⁴, NR⁴NR^{4′}, R^{4″}, OH, OR⁴, SH or SR⁴;
each X² is independently hydrogen, halogen (F, Cl, Br or I), NH₂, NHR⁴, NR⁴R^{4′}, NHOR⁴, NR⁴NR^{4′}R^{4″}, OH, OR⁴, SH or SR⁴;
each Z is CH₂, CHR¹, NH, or NHR⁴;
each R¹ is independently hydrogen, optionally substituted or unsubstituted lower alkyl, optionally substituted or unsubstituted lower alkenyl, optionally substituted or unsubstituted lower alkynyl, halogen (F, Cl, Br or I), CH₃ (Me), CH₂CH₃ (Et), or CF₃;
each R² independently is hydrogen, halogen (F, Cl, Br or I), OH, SH, OCH₃, SCH₃, NH₂, NHCH₃, CN, on N₃;
each R³ independently is hydrogen, halogen (F, Cl, Br or I), OH, SH, OCH₃, SCH₃, NH₂, NHCH₃, CN, on N₃; and
each R⁴, R^{4′}, R^{4″}, R⁵, R^{5′} and R^{5″} independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl.

In one embodiment, the carbocylic nucleoside is the β-D-enantiomer.

In another embodiment, anti-viral or anti-proliferative effective nucleoside is a nucleoside of the general formula (IV) to (V): 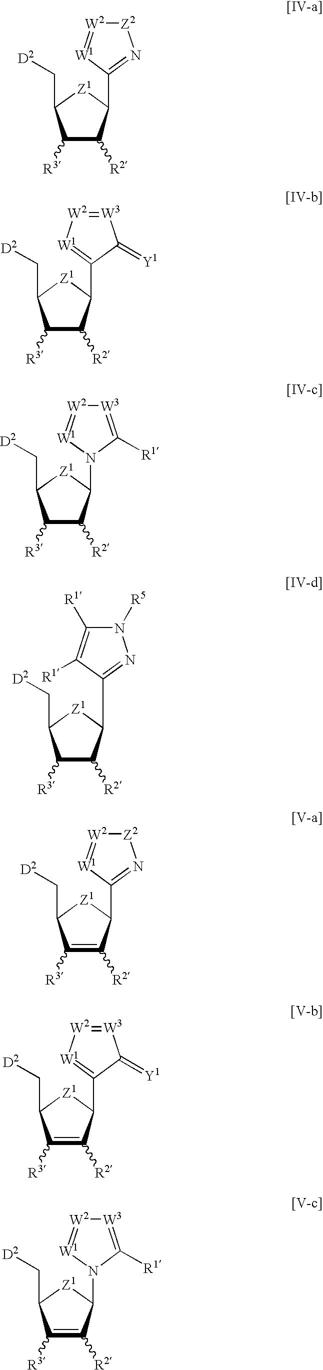 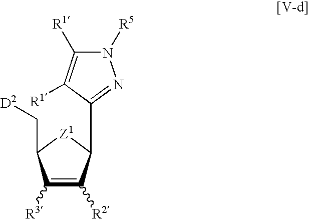
or a pharmaceutically acceptable salt and/or prodrug, including an ester, thereof, wherein:
each W¹, W², X¹, X², Z, R⁴, R^{4′}, R^{4″}, R⁵, R^{5′} and R^{5″} is the same as defined previously;
each D² is independently OD wherein D is the same as define previously, OH, SH, NH₂, or NHR⁴;
each W³ is independently N, CH, CX¹ or CR^{1′};
each R^{1′} is independently hydrogen, optionally substituted or unsubstituted lower alkyl, optionally substituted or unsubstituted lower alkenyl, optionally substituted or unsubstituted lower alkynyl, optionally substituted or unsubstituted aryl, alkylaryl, halogen (F, Cl, Br or I), CH₃ (Me), CF₃, CH₂CH₃ (Et), Pr, i-Pr, n-Bu, i-Bu, t-Bu, CH₂CN, CH₂OH, CH₂OR⁵, acyl, alkylacyl, amide, alkylamide, CH₂CO₂CH₃, CH₂C(═O)NH₂, CH₂C(═S)NH₂, C(═O)NH₂, C(═O)NHR⁵, C(═O)NR⁵R⁵, C(═S)NH₂, C(═NH)NH₂, C(═O)NHOH, C(═O)NHNH₂, alkylamine, haloalkylamine, CH₂NH₃, NH₂, NHCH₃, N(CH₃)₂, NHCH₂CH₃, NHR⁵, NR⁵R^{5′}, NHOR⁵, NR⁵NHR^{5′}, NR⁵NR^{5′}R^{5″}, OH, OCH₃, OCH₂CH₃, OR⁵, SH, SCH₃, SCH₂CH₃, SR⁵, NO₂, NO, N₃, CO₂H, CO₂R⁵, or CN;
each R^{2′} independently is hydrogen, halogen (F, Cl, Br or I), optionally substituted or unsubstituted alkyl, optionally substituted or unsubstituted lower alkyl, haloalkyl, lower haloalkyl, CH₃, CF₃, CH₂OH, CH₂F, CH₂SH, CH₂SCH₃, CH₂N₃, CH₂NH₂, OH, SH, OCH₃, SCH₃, NH₂, NHCH₃, N₃, CH═CH₂, CN, CH₂NH₂, CH₂OH, or CO₂H;
each R^{3′} independently is hydrogen, halogen (F, Cl, Br or I), optionally substituted or unsubstituted alkyl, optionally substituted or unsubstituted lower alkyl, haloalkyl, lower haloalkyl, CH₃, CF₃, CH₂OH, CH₂F, CH₂SH, CH₂SCH₃, CH₂N₃, CH₂NH₂, OH, SH, OCH₃, SCH₃, NH₂, NHCH₃, N₃, CH═CH₂, CN, CH₂NH₂, CH₂OH, or CO₂H;
each Z¹ is independently O, S, Se, CH₂, CF₂, C(═O), C(═CH₂), NH, NR⁵, or C(═Y); and
each Z² is independently O, S, Se, C(═O), C(═CH₂), NH, NR⁵, or C(═Y¹); and
each Y¹ is O, S, Se, NH, or NHR⁴;
such that there are no more than three ring-heteroatoms (i.e., no more than three O, S, N, or Se in the ring).

In one embodiment, the nucleoside is the β-D-enantiomer.

In one particular embodiment, anti-viral or anti-proliferative effective nucleoside is a β-D-nucleoside of the formula: 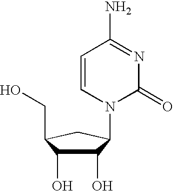
or a pharmaceutically acceptable salt and/or prodrug, including an ester, thereof.

In another particular embodiment, anti-viral or anti-proliferative effective nucleoside is a β-D-nucleoside of the formula: 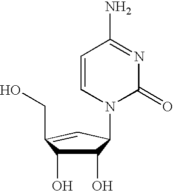
or a pharmaceutically acceptable salt and/or prodrug, including an ester, thereof.

In yet another particular embodiment, anti-viral or anti-proliferative effective nucleoside is a β-D-nucleoside of the formula: 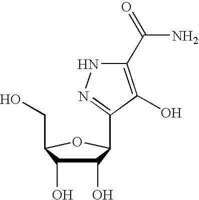
or a pharmaceutically acceptable salt and/or prodrug, including an ester, thereof.

In yet another particular embodiment, anti-viral or anti-proliferative effective nucleoside is a β-D-nucleoside of the formula: 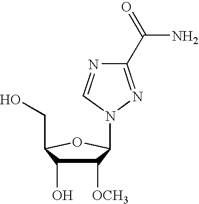
or a pharmaceutically acceptable salt and/or prodrug, including an ester, thereof.

In another embodiment, anti-viral or anti-proliferative effective agent is N-(phosphonoacetyl)-L-aspartate (PALA), or its pharmaceutically acceptable salt and/or prodrug.

### BRIEF DESCRIPTION OF THE FIGURES

FIG. 1 provides the structure of various non-limiting examples of anti-viral or anti-proliferative effective agents of the present invention, as well as the known anti-viral or anti-proliferative effective nucleosides, ribavirin, 2′-C-methyl-ribofuranyl cytosine (2C—CH₃—C), and 2′-C-methyl-ribofuranyl adenosine (2C—CH₃-A), which are used as comparative examples in the text.

FIG. 2 is a line graph illustrating the dynamics of HCV replicon containing Huh7 cell growth. HCV replicon cells were seeded at approximately 10⁵ cells per well in a 6-well plate. Over a 14-day period, cells were harvested and counted daily, and rRNA and HCV RNA were quantified by Q-RT-PCR. ▪: rRNA; ∇: HCV RNA; ●: cell count. The curves shown are averages of at least 3 different experiments.

FIG. 3 are line graphs illustrating the reduction in HCV RNA and rRNA as a function of administered dose. HCV replicon cells were seeded in the presence of test compound at approximately 10³ cells per well in a 96-well plate and incubated for 96 hours. rRNA and HCV RNA were quantified by Q-RT-PCR. ●: HCV RNA levels; ◯: rRNA levels; ▾: HCV RNA levels after correction (=subtraction of rRNA) for cellular toxicity. A: 2′-C—CH₃—C; B: ribavirin; C: CP-C; D: 3DU; E: CPE-C; F: dFdC. The plots shown are the mean results of at least three independent experiments. EC₉₀ values as given in Table 1 are read from the HCV curves corrected for cellular toxicity.

FIG. 4 are line graphs illustrating the dynamics of cell growth and HCV RNA levels after exposure to anti-HCV compounds. HCV replicon cells were seeded at approximately 10⁴ cells per well in a 24-well plate. Over a 7-day period, cells were harvested and counted daily, and rRNA and HCV RNA were quantified by Q-RT-PCR. A: IFN-α-2a at 100 IU/ml; B: ribavirin at 100 μM; C: 2′-C—CH₃—C at 100 μM; D: 2′-C—CH₃-A at 20 μM. ●: cell proliferation in absence of compound; ◯: cell proliferation in presence of compound; ▾: HCV RNA levels in untreated cells; ∇: HCV RNA levels in the presence of compound. The curves shown are averages of at least 3 different experiments.

FIG. 5 are line graphs illustrating the dynamics of the cell growth and HCV RNA levels after exposure to selected antimetabolites. Experimental set-up was identical as in FIG. 4. A: dFdC at 1 μM; B: 3-DU at 100 μM; C: CP-C at 25 μM; D: CPE-C at 2.5 μM; [symbol] : cell proliferation in absence of compound; ◯: cell proliferation in presence of compound; [symbol] : HCV RNA levels in untreated cells; ∇: HCV RNA levels in the presence of compound. The curves shown are averages of at least 3 different experiments.

FIG. 6 are line graphs illustrating the dose-response and dynamics of the cell growth and HCV RNA levels after exposure to PALA and pyrazofurin. Experimental set-up was identical as in FIG. 4.

FIG. 7 is a schematic that illustrates the biochemical pathway for de novo pyrimidine synthesis. The catalytic steps of the different enzymes are indicated by arrows, e.g. aspartate carbamoyltransferase: EC 2.1.3.2; dihydroorotase: EC 3.5.2.3; orotate reductase: EC 1.3.1.14; dihydroorotate oxidase: EC 1.3.3.1; dihydroorotate dehydrogenase: EC 1.3.99.11; orotate phosphoribosyltransferase: EC 2.4.2.10; orotidine-5′-monophosphate decarboxylase: EC 4.1.1.23; CTP synthetase: E.C. 6.3.4.2.

### DETAILED DESCRIPTION OF THE INVENTION

The present invention provides a nucleoside of formula (I)-(V) or its pharmaceutically acceptable salt and/or prodrug, including an ester, for the treatment of a host infected with a Flaviviridae, including flaviviruses, pestiviruses, or hepaciviruses, such as HCV. Alternatively, the β-D or β-L nucleoside (I)-(V) or its pharmaceutically acceptable salt and/or prodrug, including an ester, can be used for the treatment of abnormal cellular proliferation.

The present invention also provides an anti-viral or anti-proliferative effective agent, N-(phosphonoacetyl)-L-aspartate (PALA), or its pharmaceutically acceptable salt and/or prodrug, for the treatment of a host infected with a Flaviviridae, including flaviviruses, pestiviruses, or hepaciviruses, such as HCV. Alternatively, PALA, or its pharmaceutically acceptable salt or prodrug, can be used for the treatment of abnormal cellular proliferation.

In one embodiment, a method for the treatment or prophylaxis of a Flaviviridae infection, including flavivirus, pestivirus, or hepacivirus, such as HCV, as well as abnormal cellular proliferation, which includes the administration of an anti-viral or anti-proliferative effective amount of an agent of the present invention, or its pharmaceutically acceptable salt and/or prodrug, including an ester, is provided.

In another embodiment, a method for the treatment or prophylaxis of a Flaviviridae infection that includes the administration of an antiviral amount of an agent of the present invention, or its pharmaceutically acceptable salt and/or prodrug, including an ester, is provided.

In another embodiment, a method for the treatment or prophylaxis of a disease characterized by abnormal cellular proliferation that includes the administration of an anti-proliferative effective amount of an agent of the present invention, or its pharmaceutically acceptable salt and/or prodrug, including an ester, is provided.

In another embodiment, the invention is the use of one of the compounds described herein, or its pharmaceutically acceptable salt and/or prodrug, including an ester, in the treatment of a host exhibiting a viral infection or abnormal cellular proliferation, as provided herein.

In another embodiment, the invention is the use of one of the compounds described herein, or its pharmaceutically acceptable salt and/or prodrug, including an ester, in the manufacture of a medicament for the treatment of a viral infection or abnormal cellular proliferation, as provided herein.

In another embodiment, a pharmaceutical composition that includes an antiviral or anti-proliferative effective amount of an agent of the present invention, or its pharmaceutically acceptable salt and/or prodrug, including an ester, together with a pharmaceutically acceptable carrier or diluent, according to the present invention, is provided.

In another embodiment, a pharmaceutical composition with an agent of the present invention, or its pharmaceutically acceptable salt and/or prodrug, including an ester, in combination with one or more other antiviral or anti-proliferative effective agents, is provided.

In an additional embodiment, a method of treating a mammal having a virus-associated disorder which comprises administering to the mammal a pharmaceutically effective amount of an agent of the present invention, or a pharmaceutically acceptable salt and/or prodrug, including an ester, thereof, is provided.

In an additional embodiment, a method of treating a mammal having disorder associated with abnormal cellular proliferation, which comprises administering to the mammal a pharmaceutically effective amount of an agent of the present invention, or a pharmaceutically acceptable salt and/or prodrug, including an ester, thereof, is provided.

In particular, the invention includes the described compounds, and their pharmaceutically acceptable salts and/or prodrug, including an ester,s, in methods for treating or preventing, or uses for the treatment or prophylaxis of, or uses in the manufacture of a medicament for the treatment or prophylaxis of the following:
(a) a Flaviviridae infection, including all members of the Hepacivirus genus (HCV), Pestivirus genus (BVDV, CSFV, BDV), or Flavivirus genus (Dengue virus, Japanese encephalitis virus group (including West Nile Virus), and Yellow Fever virus); and
(b) abnormal cellular proliferation, including malignant tumors.

In one aspect of the present invention, antimetabolites for several nucleotide biosynthetic pathways were evaluated for their anti-replicon activity and molecular toxicity in Huh7 cells stably transfected with a bicistronic subgenomic HCV replicon and were found to possess anti-HCV activity. This activity was evaluated by quantifying both HCV RNA levels and rRNA levels simultaneously, and by studying the dynamics of cell growth in relation to the HCV RNA copy numbers per cell.

The parameters for a specific antiviral effect in HCV replicon cells are defined as follows: the test compound should (i) not or only minimally interfere with the obligatory exponential cell growth, (ii) not or only minimally reduce cellular host RNA levels, and (iii) reduce the HCV RNA copy number per cell, as compared to the control experiment and the pretreatment sample.

Without being constrained by theory, while certain tested antimetabolites caused a cytostatic effect on the cell growth dynamics, a high reduction of HCV RNA copies per cell was seen with several de novo ribo-pyrimidine synthesis inhibitors (e.g., dFdC, CP-C, CPE-C, 3DU, PALA, and pyrazofurin); certain other antimetabolites, such as IMPDH inhibitors (e.g., ribavirin, tiazofurin, mycophenolic acid, C2-MAD), ribonucleotide reductase inhibitors (e.g., tezacytabine, deferoxamine) and thymidylate synthase inhibitors (e.g., 2′-deoxy-5FU), can show antiviral effects, but when corrected for the reduction in cellular RNA levels, specificity may be significantly decreased. Therefore, antimetabolites of the de novo ribo-pyrimidine pathway may mimic the observation seen in confluent replicon cells, namely cytostasis combined with a sharp decrease in replicon copy number per cell.

Compounds of the Invention

In one embodiment of the invention, the anti-viral or anti-proliferative effective nucleoside is a carbocyclic nucleoside of the general formula (I) to (II): 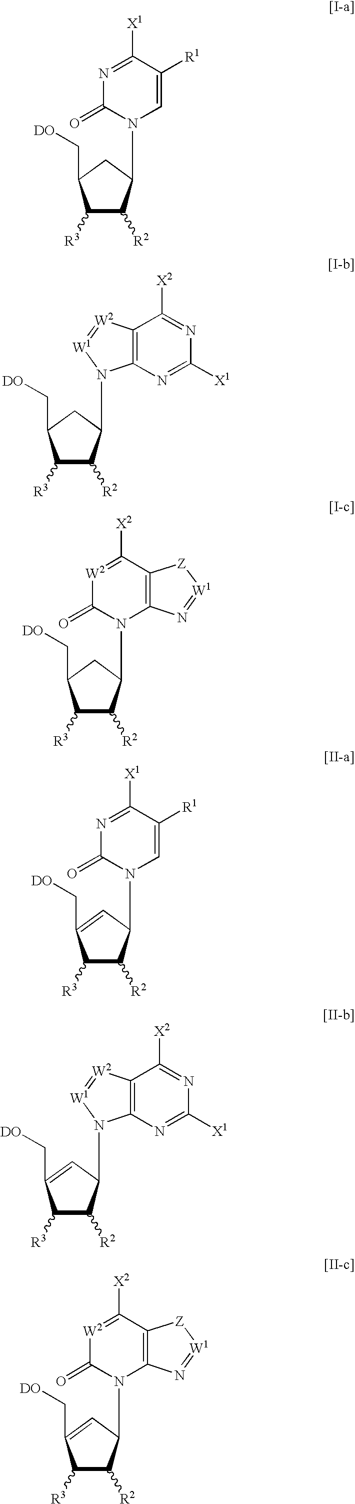
or a pharmaceutically acceptable salt and/or prodrug, including an ester, thereof, wherein:
each D is hydrogen, alkyl, acyl, monophosphate, diphosphate, triphosphate, monophosphate ester, diphosphate ester, triphosphate ester, phospholipid or amino acid;
each W¹ and W² is independently N, CH, CX² or CR¹;
each X¹ is independently NH₂, NHR⁴, NR⁴R^{4′}, NHOR⁴, NR⁴NR^{4′}R^{4″}, OH, OR⁴, SH or SR⁴;
each X² is independently hydrogen, halogen (F, Cl, Br or I), NH₂, NHR⁴, NR⁴R^{4′}, NHOR⁴, NR⁴NR^{4′}R^{4″}, OH, OR⁴, SH or SR⁴;
each Z is CH₂, CHR¹, NH, or NHR⁴;
each R¹ is independently hydrogen, optionally substituted or unsubstituted lower alkyl, optionally substituted or unsubstituted lower alkenyl, optionally substituted or unsubstituted lower alkynyl, halogen (F, Cl, Br or I), CH₃ (Me), CH₂CH₃ (Et), or CF₃;
each R² independently is hydrogen, halogen (F, Cl, Br or I), OH, SH, OCH₃, SCH₃, NH₂, NHCH₃, CN, on N₃;
each R³ independently is hydrogen, halogen (F, Cl, Br or I), OH, SH, OCH₃, SCH₃, NH₂, NHCH₃, CN, on N₃; and
each R⁴, R^{4′}, R^{4″}, R⁵, R^{5′} and R^{5″} independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl.

In one embodiment, the carbocylic nucleoside is the β-D-enantiomer.

In another embodiment, anti-viral or anti-proliferative effective nucleoside is a nucleoside of the general formula (IV) to (V): 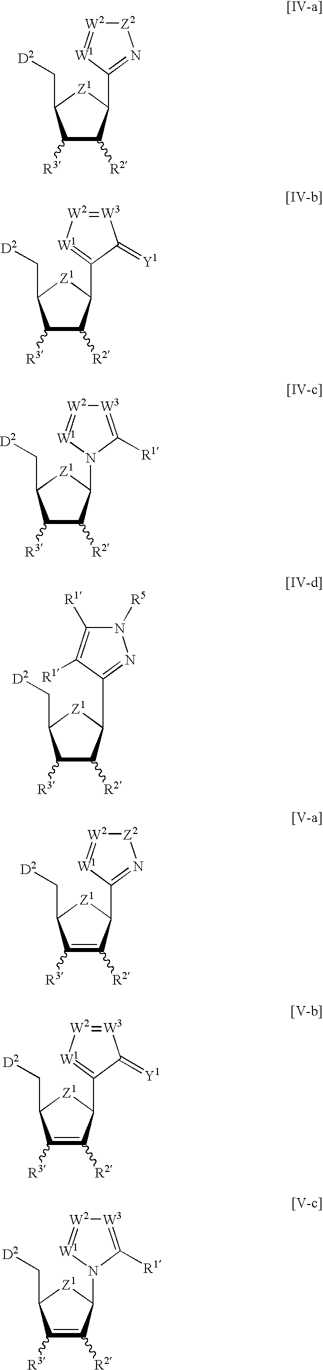 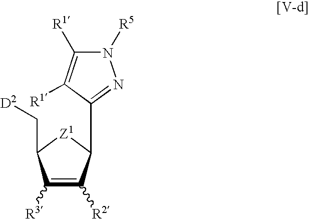
or a pharmaceutically acceptable salt and/or prodrug, including an ester, thereof, wherein:
each W¹, W², X¹, X², Z, R⁴, R^{4′}, R^{4″}, R⁵, R^{5′} and R^{5″} is the same as defined previously;
each D² is independently OD wherein D is the same as define previously, OH, SH, NH₂, or NHR⁴;
each W³ is independently N, CH, CX¹ or CR^{1′};
each R^{1′} is independently hydrogen, optionally substituted or unsubstituted lower alkyl, optionally substituted or unsubstituted lower alkenyl, optionally substituted or unsubstituted lower alkynyl, optionally substituted or unsubstituted aryl, alkylaryl, halogen (F, Cl, Br or I), CH₃ (Me), CF₃, CH₂CH₃ (Et), Pr, i-Pr, n-Bu, i-Bu, t-Bu, CH₂CN, CH₂OH, CH₂OR⁵, acyl, alkylacyl, amide, alkylamide, CH₂CO₂CH₃, CH₂C(═O)NH₂, CH₂C(═S)NH₂, C(═O)NH₂, C(═O)NHR⁵, C(═O)NR⁵R⁵, C(═S)NH₂, C(═NH)NH₂, C(═O)NHOH, C(═O)NHNH₂, alkylamine, haloalkylamine, CH₂NH₃, NH₂, NHCH₃, N(CH₃)₂, NHCH₂CH₃, NHR⁵, NR⁵R^{5′}, NHOR⁵, NR⁵NHR^{5′}, NR⁵NR^{5′}R^{5″}, OH, OCH₃, OCH₂CH₃, OR⁵, SH, SCH₃, SCH₂CH₃, SR⁵, NO₂, NO, N₃, CO₂H, CO₂R⁵, or CN;
each R^{2′} independently is hydrogen, halogen (F, Cl, Br or I), optionally substituted or unsubstituted alkyl, optionally substituted or unsubstituted lower alkyl, haloalkyl, lower haloalkyl, CH₃, CF₃, CH₂OH, CH₂F, CH₂SH, CH₂SCH₃, CH₂N₃, CH₂NH₂, OH, SH, OCH₃, SCH₃, NH₂, NHCH₃, N₃, CH═CH₂, CN, CH₂NH₂, CH₂OH, or CO₂H;
each R^{3′} independently is hydrogen, halogen (F, Cl, Br or I), optionally substituted or unsubstituted alkyl, optionally substituted or unsubstituted lower alkyl, haloalkyl, lower haloalkyl, CH₃, CF₃, CH₂OH, CH₂F, CH₂SH, CH₂SCH₃, CH₂N₃, CH₂NH₂, OH, SH, OCH₃, SCH₃, NH₂, NHCH₃, N₃, CH═CH₂, CN, CH₂NH₂, CH₂OH, or CO₂H;
each Z¹ is independently O, S, Se, CH₂, CF₂, C(═O), C(═CH₂), NH, NR⁵, or C(═Y); and
each Z² is independently O, S, Se, C(═O), C(═CH₂), NH, NR⁵, or C(═Y¹); and
each Y¹ is O, S, Se, NH, or NHR⁴;
such that there are no more than three ring-heteroatoms (i.e., no more than three O, S, N, or Se in the ring).

In one embodiment, the nucleoside is the β-D-enantiomer.

In a particular embodiment, Z¹ is O. In another embodiment, Z¹ is S. In yet another embodiment, Z¹ is CH₂. In yet another embodiment, Z¹ is CF₂.

In one sub-embodiment, anti-viral or anti-proliferative effective nucleoside is a β-D-nucleoside of the general formula (IV-a*): 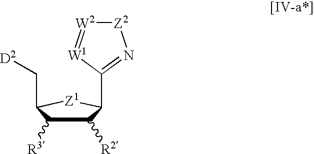
or a pharmaceutically acceptable salt and/or prodrug, including an ester, thereof, wherein:
each D² is independently OD wherein D is the same as define previously, OH, SH, NH₂, or NHR⁴;
each Z¹ is independently O, S, CH₂, CF₂, C(═O), or C(═CH₂);
each Z² is independently O, S, Se, C(═O), C(═S), C(═CH₂), NH, or NR⁵;
each W¹ and W² is independently N or CR;
each R^{1′} is independently hydrogen, halogen (F, Cl, Br or I), CH₃ (Me), CH₂CH₃ (Et), Pr, i-Pr, n-Bu, i-Bu, t-Bu, CH₂CN, CH₂CO₂CH₃, CH₂C(═O)NH₂, CH₂C(═S)NH₂, C(═O)NH₂, C(═S)NH₂, C(═NH)NH₂, C(═O)NHOH, C(═O)NHNH₂, CH₂NH₃, NH₂, NHCH₃, N(CH₃)₂, NHCH₂CH₃, OH, OCH₃, OCH₂CH₃, SH, SCH₃, SCH₂CH₃, CO₂H, CN, or CHR*NH₂;
each R* is hydrogen or halogen (F, Cl, Br, or I);
each R^{2′} independently is hydrogen, halogen (F, Cl, Br or I), CH₃, CH₂OH, CH₂F, CH₂SH, CH₂SCH₃, CH₂N₃, CH₂NH₂, OH, OCH₃, or NH₂;
each R^{3′} independently is hydrogen, halogen (F, Cl, Br or I), CH₃, CH₂OH, CH₂F, CH₂SH, CH₂SCH₃, CH₂N₃, CH₂NH₂, OH, OCH₃, or NH₂;
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl; and
each R⁵ is independently hydrogen, CH₃ (Me), CH₂CH₃ (Et), Pr, i-Pr, n-Bu, i-Bu, t-Bu, CH₂CN, CH₂CO₂CH₃, CH₂C(═O)NH₂, CH₂C(═S)NH₂, C(═O)NH₂, or C(═S)NH₂;
such that there are no more than three ring-heteroatoms (i.e., no more than three O, S, N, or Se in the ring).

In a particular embodiment, Z¹ is O. In another embodiment, Z¹ is S. In yet another embodiment, Z¹ is CH₂. In still another embodiment, Z¹ is CF₂.

In another sub-embodiment, anti-viral or anti-proliferative effective nucleoside is a β-D-nucleoside of the general formula (IV-b*): 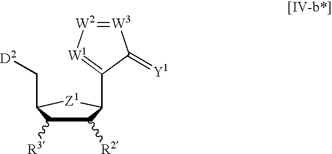
or a pharmaceutically acceptable salt and/or prodrug, including an ester, thereof, wherein:
each D² is independently OD wherein D is the same as define previously, OH, SH, NH₂, or NHR⁴;
each Z¹ is independently O, S, CH₂, CF₂, C(═O), or C(═CH₂);
each Y¹ is independently O, S, Se, or NH;
each W¹ and W² is independently N or CR^{1′};
each W³ is independently N, CH, CCH₃, CF, CCl, CBr, Cl, CCO₂H, CCO₂CH₃, CCONH₂, CC(═S)NH₂, or CCN;
each R^{1′} is independently hydrogen, halogen (F, Cl, Br or I), CH₃ (Me), CH₂CH₃ (Et), Pr, i-Pr, n-Bu, i-Bu, t-Bu, CH₂CN, CH₂CO₂CH₃, CH₂C(═O)NH₂, CH₂C(═S)NH₂, C(═O)NH₂, C(═S)NH₂, NH₂, NHCH₃, N(CH₃)₂, NHCH₂CH₃, OH, OCH₃, OCH₂CH₃, SH, SCH₃, SCH₂CH₃, CO₂H, or CN;
each R^{2′} independently is hydrogen, halogen (F, Cl, Br or I), CH₃, CH₂OH, CH₂F, CH₂SH, CH₂SCH₃, CH₂N₃, CH₂NH₂, OH, OCH₃, or NH₂;
each R^{3′} independently is hydrogen, halogen (F, Cl, Br or I), CH₃, CH₂OH, CH₂F, CH₂SH, CH₂SCH₃, CH₂N₃, CH₂NH₂, OH, OCH₃, or NH₂; and
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl.

In a particular embodiment, Z¹ is O. In another embodiment, Z¹ is S. In yet another embodiment, Z¹ is CH₂. In yet another embodiment, Z¹ is CF₂.

In yet another sub-embodiment, anti-viral or anti-proliferative effective nucleoside is a β-D-nucleoside of the general formula (IV-c*): 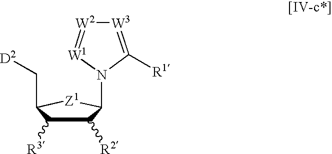
or a pharmaceutically acceptable salt and/or prodrug, including an ester, thereof, wherein:
each D² is independently OD wherein D is the same as define previously, OH, SH, NH₂, or NHR⁴;
each Z¹ is independently O, S, CH₂, CF₂, C(═O), or C(═CH₂);
each Y¹ is independently O, S, Se, or NH;
each W¹, W², and W³ is independently N or CR^{1′};
each R^{1′} is independently hydrogen, halogen (F, Cl, Br or I), CH₃(Me), CH₂CH₃ (Et), Pr, i-Pr, n-Bu, i-Bu, t-Bu, CH₂CN, CH₂CO₂CH₃, CH₂C(═O)NH₂, CH₂C(═S)NH₂, C(═O)NH₂, C(═S)NH₂, NH₂, NHCH₃, N(CH₃)₂, NHCH₂CH₃, OH, OCH₃, OCH₂CH₃, SH, SCH₃, SCH₂CH₃, CO₂H, or CN;
each R^{2′} independently is hydrogen, halogen (F, Cl, Br or I), CH₃, CH₂OH, CH₂F, CH₂SH, CH₂SCH₃, CH₂N₃, CH₂NH₂, OH, OCH₃, or NH₂;
each R^{3′} independently is hydrogen, halogen (F, Cl, Br or I), CH₃, CH₂OH, CH₂F, CH₂SH, CH₂SCH₃, CH₂N₃, CH₂NH₂, OH, OCH₃, or NH₂; and
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl.

In a particular embodiment, Z¹ is O. In another embodiment, Z¹ is S. In yet another embodiment, Z¹ is CH₂. In yet another embodiment, Z¹ is CF₂.

In yet another sub-embodiment, anti-viral or anti-proliferative effective nucleoside is a β-D-nucleoside of the general formula (IV-d*): 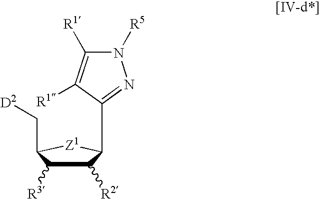
or a pharmaceutically acceptable salt and/or prodrug, including an ester, thereof, wherein:
each D² is independently OD wherein D is the same as define previously, OH, SH, NH₂, or NHR⁴;
each Z¹ is independently O, S, CH₂, CF₂, C(═O), or C(═CH₂);
each R^{1′} is independently CN, CO₂CH₃, C(═O)NH₂, C(═S)NH₂, or C(═NH)NH₂;
each R^{1″} is independently OH, SH, NH₂, or NHR⁵;
each R^{2′} independently is hydrogen or halogen (F, Cl, Br or I), CH₃, CH₂OH, CH₂F, CH₂SH, CH₂SCH₃, CH₂N₃, CH₂NH₂, OH, OCH₃, or NH₂;
each R^{3′} independently is hydrogen or halogen (F, Cl, Br or I), CH₃, CH₂OH, CH₂F, CH₂SH, CH₂SCH₃, CH₂N₃, CH₂NH₂, OH, OCH₃, or NH₂;
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl; and
each R⁵ is independently is hydrogen, optionally substituted or unsubstituted lower alkyl, or an optionally substituted or unsubstituted acyl.

In a particular embodiment, Z¹ is O. In another embodiment, Z¹ is S. In yet another embodiment, Z¹ is CH₂. In yet another embodiment, Z¹ is CF₂.

In one particular embodiment, anti-viral or anti-proliferative effective nucleoside is a β-D-nucleoside of the formula: 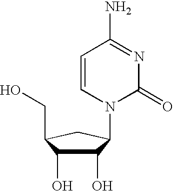
or a pharmaceutically acceptable salt and/or prodrug, including an ester, thereof.

In another particular embodiment, anti-viral or anti-proliferative effective nucleoside is a β-D-nucleoside of the formula: 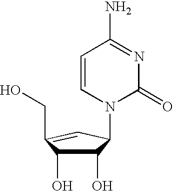
or a pharmaceutically acceptable salt and/or prodrug, including an ester, thereof.

In yet another particular embodiment, anti-viral or anti-proliferative effective nucleoside is a β-D-nucleoside of the formula: 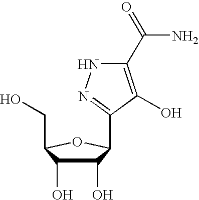
or a pharmaceutically acceptable salt and/or prodrug, including an ester, thereof.

In yet another particular embodiment, anti-viral or anti-proliferative effective nucleoside is a β-D-nucleoside of the formula: 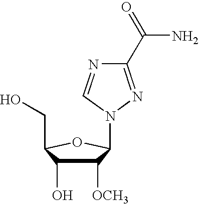
or a pharmaceutically acceptable salt and/or prodrug, including an ester, thereof.

In another embodiment, anti-viral or anti-proliferative effective agent is N-(phosphonoacetyl)-L-aspartate (PALA), or its pharmaceutically acceptable salt and/or prodrug.

Stereoisomerism and Polymorphism

Compounds of the present invention having a chiral center may exist in and be isolated in optically active and racemic forms. Some compounds may exhibit polymorphism. The present invention encompasses racemic, optically-active, polymorphic, or stereoisomeric form, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution.

As shown below, a nucleoside contains at least two critical chiral carbon atoms (*). In general, the substituents on the chiral carbons [the specified purine or pyrimidine base (referred to as the Cl substituent when using the sugar ring intermediate numbering) and CH₂OH (referred to as the C4 substituent)] of the nucleoside can be either cis (on the same side) or trans (on opposite sides) with respect to the sugar ring system. Both the cis and trans racemates consist of a pair of optical isomers. Hence, each compound has four individual stereoisomers. The four stereoisomers are represented by the following configurations (when orienting the sugar moiety in a horizontal plane such that the —O— moiety is in back): (1) cis, with both groups “up”, which is referred to as β-D; (2) the mirror image, i.e., cis, with both groups “down”, which is the mirror image is referred to as β-L; (3) trans with the C4 substituent “up” and the C1 substituent “down” (referred to as α-D); and (4) trans with the C4 substituent “down” and the C1 substituent “up” (referred to as α-L). The two cis enantiomers together are referred to as a racemic mixture of β-enantiomers, and the two trans enantiomers are referred to as a racemic mixture of α-enantiomers.

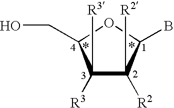

The four possible stereoisomers of the claimed compounds are illustrated below. 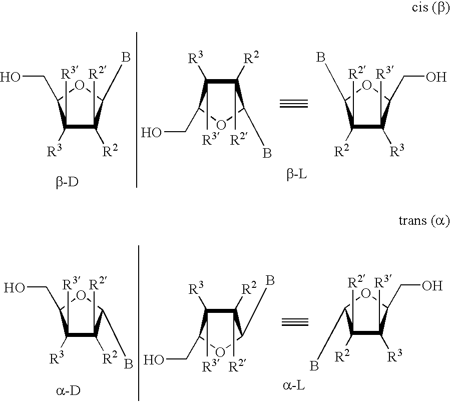
Definitions

The term “alkyl,” as used herein, unless otherwise specified, refers to a saturated straight, branched, or cyclic, primary, secondary, or tertiary hydrocarbon, including but not limited to those of C₁ to C₁₀, and specifically includes lower alkyl, such as methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The alkyl group can be optionally substituted with one or more moieties selected from the group consisting of alkyl, halo (e.g. CH₂F or CF₃), haloalkyl, hydroxyl, carboxyl, acyl, acyloxy, amino, amido, carboxyl derivatives, alkylamino, dialkylamino, arylamino, alkoxy, aryloxy, nitro, cyano, azido, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl,” as used herein, and unless otherwise specified, refers to a C₁ to C₄ saturated straight, branched, or if appropriate, a cyclic (for example, cyclopropyl) alkyl group, including both substituted and unsubstituted forms. Non-limiting examples include methyl, trifluoromethyl, ethyl, propyl, isopropyl, cyclopropyl, butyl, isobutyl, and t-butyl.

The term “alkylene” or “alkenyl” refers to a saturated hydrocarbyldiyl radical of straight or branched configuration, including but not limited to those that have from two to ten carbon atoms. Included within the scope of this term are methylene, 1,2-ethane-diyl, 1,1-ethane-diyl, 1,3-propane-diyl, 1,2-propane-diyl, 1,3-butane-diyl, 1,4-butane-diyl and the like. The alkylene group or other divalent moiety disclosed herein can be optionally substituted with one or more moieties selected from the group consisting of alkyl, halo, haloalkyl, hydroxyl, carboxyl, acyl, acyloxy, amino, amido, carboxyl derivatives, alkylamino, azido, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

As used herein, the term “alkynyl,” unless otherwise specified, includes a straight chain or branched, acyclic hydrocarbon having at least 2 carbon atoms and including at least one carbon-carbon triple bond. Examples of alkynyl include, but are not limited to, acetylenyl, propynyl, 1-butynyl, 2-butynyl, 1-pentynyl, 2-pentynyl, 3-methyl-1-butynyl, 4-pentynyl, 1-hexynyl, 2-hecynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, and 9-decynyl moieties.

The term “aryl,” as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of bromo, chloro, fluoro, iodo, hydroxyl, azido, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “aralkyl,” as used herein, and unless otherwise specified, refers to an aryl group as defined above linked to the molecule through an alkyl group as defined above. The term “alkaryl” or “alkylaryl” as used herein, and unless otherwise specified, refers to an alkyl group as defined above linked to the molecule through an aryl group as defined above. In each of these groups, the alkyl group can be optionally substituted as describe above and the aryl group can be optionally substituted with one or more moieties selected from the group consisting of alkyl, halo, haloalkyl, hydroxyl, carboxyl, acyl, acyloxy, amino, amido, azido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included within the scope of the term aryl are phenyl; naphthyl; phenylmethyl; phenylethyl; 3,4,5-trihydroxyphenyl; 3,4,5-trimethoxyphenyl; 3,4,5-triethoxy-phenyl; 4-chlorophenyl; 4-methylphenyl; 3,5-di-tertiarybutyl-4-hydroxyphenyl; 4-fluorophenyl; 4-chloro-1-naphthyl; 2-methyl-1-naphthylmethyl; 2-naphthylmethyl; 4-chlorophenylmethyl; 4-t-butylphenyl; 4-t-butylphenylmethyl and the like.

The term “alkylamino” or “arylamino” refers to an amino group that has one or two alkyl or aryl substituents, respectively.

The term “halogen,” as used herein, includes fluorine, chlorine, bromine and iodine.

The term “enantiomerically enriched” is used throughout the specification to describe a nucleoside which includes at least about 95%, preferably at least 96%, more preferably at least 97%, even more preferably, at least 98%, and even more preferably at least about 99% or more of a single enantiomer of that nucleoside. When a nucleoside of a particular configuration (D or L) is referred to in this specification, it is presumed that the nucleoside is an enantiomerically enriched nucleoside, unless otherwise stated.

Relative to viral infection, the term “host,” as used herein, refers to a unicellular or multicellular organism in which the virus can replicate, including cell lines and animals, and preferably a human. Alternatively, the host can be carrying a part of the viral genome, whose replication or function can be altered by the compounds of the present invention. The term host specifically refers to infected cells, cells transfected with all or part of the viral genome and animals, in particular, primates (including chimpanzees) and humans.

Relative to abnormal cellular proliferation, the term “host” refers to unicellular or multicellular organism in which abnormal cellular proliferation can be mimicked. The term host specifically refers to cells that abnormally proliferate, either from natural or unnatural causes (for example, from genetic mutation or genetic engineering, respectively), and animals, in particular, primates (including chimpanzees) and humans. In most animal applications of the present invention, the host is a human patient. Veterinary applications, in certain indications, however, are clearly anticipated by the present invention (such as bovine viral diarrhea virus in cattle, hog cholera virus in pigs, and border disease virus in sheep).

The term “pharmaceutically acceptable salt or prodrug” is used throughout the specification to describe any pharmaceutically acceptable form (such as an ester, phosphate ester, salt of an ester or a related group) of a compound which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

Pharmaceutically Acceptable Salts and Prodrugs

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the nucleosides described herein can be administered as a nucleotide prodrug to increase the activity, bioavailability, stability or otherwise alter the properties of the nucleoside. A number of nucleotide prodrug ligands are known. In general, alkylation, acylation or other lipophilic modification of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed nucleosides to achieve a desired effect.

The active nucleoside can also be provided as a 5′-phosphoether lipid or a 5′-ether lipid, as disclosed in the following references, which are incorporated by reference herein: Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine.” Antimicrob. Agents Chemother. 36:2025.2029; Hosetler, K. Y., L. M. Stuhmiller, H. B. Lenting, H. van den Bosch, and D. D. Richman, 1990. “Synthesis and antiretroviral activity of phospholipid analogs of azidothymidine and other antiviral nucleosides.” J. Biol. Chem. 265:61127.

In one embodiment, the active nucleoside is be provided as a SATE prodrug.

Nonlimiting examples of U.S. patents that disclose suitable lipophilic substituents that can be covalently incorporated into the nucleoside, preferably at the 5′-OH position of the nucleoside or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

Pharmaceutical Compositions

Pharmaceutical compositions based upon a β-D or β-L compound of formula (I)-(V) or PALA, or its pharmaceutically acceptable salt and/or prodrug, including an ester, can be prepared in a therapeutically effective amount for any of the indications described herein, including a Flaviviridae viral infection or abnormal cellular proliferation, optionally in combination with a pharmaceutically acceptable additive, carrier or excipient. The therapeutically effective amount may vary with the infection or condition to be treated, its severity, the treatment regimen to be employed, the pharmacokinetics of the agent used, as well as the patient treated.

In one aspect according to the present invention, the compound according to the present invention is formulated preferably in admixture with a pharmaceutically acceptable carrier. In general, it is preferable to administer the pharmaceutical composition in orally administrable form, but formulations may be administered via parenteral, intravenous, intramuscular, transdermal, buccal, subcutaneous, suppository or other route. Intravenous and intramuscular formulations are preferably administered in sterile saline. One of ordinary skill in the art may modify the formulation within the teachings of the specification to provide numerous formulations for a particular route of administration without rendering the compositions of the present invention unstable or compromising its therapeutic activity. In particular, a modification of a desired compound to render it more soluble in water or other vehicle, for example, may be easily accomplished by routine modification (salt formulation, esterification, etc.).

In certain pharmaceutical dosage forms, the prodrug form of the compound, especially including acylated (acetylated or other) and ether derivatives, phosphate esters and various salt forms of the present compounds, is preferred. One of ordinary skill in the art will recognize how to readily modify the present compound to a prodrug form to facilitate delivery of active compound to a targeted site within the host organism or patient. The artisan also will take advantage of favorable pharmacokinetic parameters of the prodrug form, where applicable, in delivering the desired compound to a targeted site within the host organism or patient to maximize the intended effect of the compound in the treatment of Flaviviridae (including HCV) infections or conditions related to abnormal cellular proliferation.

The amount of compound included within therapeutically active formulations, according to the present invention, is an effective amount for treating the infection or condition, in preferred embodiments, a Flaviviridae (including HCV) infection or a condition related to abnormal cellular proliferation. In general, a therapeutically effective amount of the present compound in pharmaceutical dosage form usually ranges from about 0.1 mg/kg to about 100 mg/kg or more, depending upon the compound used, the condition or infection treated and the route of administration. For purposes of the present invention, a prophylactically or preventively effective amount of the compositions, according to the present invention, falls within the same concentration range as set forth above for therapeutically effective amount and is usually the same as a therapeutically effective amount.

Administration of the active compound may range from continuous (intravenous drip) to several oral administrations per day (for example, Q.I.D., B.I.D., etc.) and may include oral, topical, parenteral, intramuscular, intravenous, subcutaneous, transdermal (which may include a penetration enhancement agent), buccal and suppository administration, among other routes of administration. Enteric-coated oral tablets may also be used to enhance bioavailability and stability of the compounds from an oral route of administration. The most effective dosage form will depend upon the pharmacokinetics of the particular agent chosen, as well as the severity of disease in the patient. Oral dosage forms are particularly preferred, because of ease of administration and prospective favorable patient compliance.

To prepare the pharmaceutical compositions according to the present invention, a therapeutically effective amount of one or more of the compounds according to the present invention is preferably mixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated for sustained release by standard techniques. The use of these dosage forms may significantly impact the bioavailability of the compounds in the patient.

For parenteral formulations, the carrier will usually comprise sterile water or aqueous sodium chloride solution, though other ingredients, including those that aid dispersion, also may be included. Where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present invention.

In particularly preferred embodiments according to the present invention, the compounds and compositions are used to treat, prevent or delay the onset of Flaviviridae (including HCV) infections or conditions related to abnormal cellular proliferation. Preferably, to treat, prevent or delay the onset of the infection or condition, the compositions will be administered in oral dosage form in amounts ranging from about 250 micrograms up to about 1 gram or more at least once a day, preferably, or up to four times a day. The present compounds are preferably administered orally, but may be administered parenterally, topically or in suppository form.

The compounds according to the present invention, because of their low toxicity to host cells in certain instances, may be advantageously employed prophylactically to prevent Flaviviridae (including HCV) infections or conditions related to abnormal cellular proliferation or to prevent the occurrence of clinical symptoms associated with the viral infection or condition. Thus, the present invention also encompasses methods for the prophylactic treatment of viral infection, and in particular Flaviviridae (including HCV) infections or of a condition related to abnormal cellular proliferation. In this aspect, according to the present invention, the present compositions are used to prevent or delay the onset of a Flaviviridae (including HCV) infection or a condition related to abnormal cellular proliferation. This prophylactic method comprises administration to a patient in need of such treatment, or who is at risk for the development of the virus or condition, an amount of a compound according to the present invention effective for alleviating, preventing or delaying the onset of the viral infection or condition. In the prophylactic treatment according to the present invention, it is preferred that the antiviral or antiproliferative compound utilized should be low in toxicity and preferably non-toxic to the patient. It is particularly preferred in this aspect of the present invention that the compound that is used should be maximally effective against the virus or condition and should exhibit a minimum of toxicity to the patient. In the case of Flaviviridae (including HCV) infections or conditions related to abnormal cellular proliferation, compounds according to the present invention, which may be used to treat these disease states, may be administered within the same dosage range for therapeutic treatment (i.e., about 250 micrograms up to 1 gram or more from one to four times per day for an oral dosage form) as a prophylactic agent to prevent the proliferation of a Flaviviridae (including HCV) infections or conditions related to abnormal cellular proliferation, or alternatively, to prolong the onset of a Flaviviridae (including HCV) infections or conditions related to abnormal cellular proliferation, which manifests itself in clinical symptoms.

In addition, compounds according to the present invention can be administered in combination or alternation with one or more antiviral, anti-HBV, anti-HCV or anti-herpetic agent or interferon, anti-cancer or antibacterial agents, including other compounds of the present invention. Certain compounds according to the present invention may be effective for enhancing the biological activity of certain agents according to the present invention by reducing the metabolism, catabolism or inactivation of other compounds and as such, are co-administered for this intended effect.

Combination and/or Alternation Therapies for the Treatment of Flaviviridae Infection

It has been recognized that drug-resistant variants of viruses can emerge after prolonged treatment with an antiviral agent. Drug resistance most typically occurs by mutation of a gene that encodes for an enzyme used in the viral replication cycle, and most typically in the case of HCV, the RNA-dependent-RNA polymerase. It has been demonstrated that the efficacy of a drug against viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the virus.

Examples of agents that have been identified as active against the hepatitis C virus, and thus can be used in combination or alternation with one or more nucleosides of general formula (I)-(V) or PALA include:

(1) Interferon

A number of patents disclose Flaviviridae, including HCV, treatments, using interferon-based therapies. For example, U.S. Pat. No. 5,980,884 to Blatt et al. discloses methods for retreatment of patients afflicted with HCV using consensus interferon. U.S. Pat. No. 5,942,223 to Bazer et al. discloses an anti-HCV therapy using ovine or bovine interferon-tau. U.S. Pat. No. 5,928,636 to Alber et al. discloses the combination therapy of interleukin-12 and interferon alpha for the treatment of infectious diseases including HCV. U.S. Pat. No. 5,849,696 to Chretien et al. discloses the use of thymosins, alone or in combination with interferon, for treating HCV. U.S. Pat. No. 5,830,455 to Valtuena et al. discloses a combination HCV therapy employing interferon and a free radical scavenger. U.S. Pat. No. 5,738,845 to Imakawa discloses the use of human interferon tau proteins for treating HCV. Other interferon-based treatments for HCV are disclosed in U.S. Pat. No. 5,676,942 to Testa et al., U.S. Pat. No. 5,372,808 to Blatt et al., and U.S. Pat. No. 5,849,696. A number of patents also disclose pegylated forms of interferon, such as U.S. Pat. Nos. 5,747,646, 5,792,834 and 5,834,594 to Hoffmann-La Roche Inc; PCT Publication No. WO 99/32139 and WO 99/32140 to Enzon; WO 95/13090 and U.S. Pat. Nos. 5,738,846 and 5,711,944 to Schering; and U.S. Pat. No. 5,908,621 to Glue et al.

Interferon alpha-2a and interferon alpha-2b are currently approved as monotherapy for the treatment of HCV. ROFERON®-A (Roche) is the recombinant form of interferon alpha-2a. PEGASYS® (Roche) is the pegylated (i.e. polyethylene glycol modified) form of interferon alpha-2a. INTRON®A (Schering Corporation) is the recombinant form of Interferon alpha-2b, and PEG-INTRON® (Schering Corporation) is the pegylated form of interferon alpha-2b.

Other forms of interferon alpha, as well as interferon beta, gamma, tau and omega are currently in clinical development for the treatment of HCV. For example, INFERGEN (interferon alphacon-1) by InterMune, OMNIFERON (natural interferon) by Viragen, ALBUFERON by Human Genome Sciences, REBIF (interferon beta-1a) by Ares-Serono, Omega Interferon by BioMedicine, Oral Interferon Alpha by Amarillo Biosciences, and interferon gamma, interferon tau, and interferon gamma-1b by InterMune are in development.

(2) Ribavirin (Battaglia, A. M. et al., Ann. Pharmacother, 2000, 34, 487-494); Berenguer, M. et al. Antivir. Ther., 1998, 3 (Suppl. 3), 125-136).

Ribavirin (1-β-D-ribofuranosyl-1-1,2,4-triazole-3-carboxamide) is a synthetic, non-interferon-inducing, broad spectrum antiviral nucleoside analog. It is sold under the trade names Virazole™ (The Merck Index, 11th edition, Editor: Budavari, S., Merck & Co., Inc., Rahway, N.J., p1304, 1989); Rebetol (Schering Plough) and Co-Pegasus (Roche). U.S. Pat. No. 3,798,209 and RE29,835 (ICN Pharmaceuticals) disclose and claim ribavirin. Ribavirin is structurally similar to guanosine, and has in vitro activity against several DNA and RNA viruses including Flaviviridae (Gary L. Davis. Gastroenterology 118:S104-S114, 2000). U.S. Pat. No. 4,211,771 (to ICN Pharmaceuticals) discloses the use of ribavirin as an antiviral agent. Ribavirin reduces serum amino transferase levels to normal in 40% of patients, but it does not lower serum levels of HCV-RNA (Gary L. Davis. Gastroenterology 118:S104-S114, 2000). Thus, ribavirin alone is not effective in reducing viral RNA levels. Additionally, ribavirin has significant toxicity and is known to induce anemia.

Combination of Interferon and Ribavirin

The current standard of care for chronic hepatitis C is combination therapy with an alpha interferon and ribavirin. The combination of interferon and ribavirin for the treatment of HCV infection has been reported to be effective in the treatment of interferon naïve patients (Battaglia, A. M. et al., Ann. Pharmacother. 34:487-494, 2000), as well as for treatment of patients when histological disease is present (Berenguer, M. et al. Antivir. Ther. 3(Suppl. 3):125-136, 1998). Studies have show that more patients with hepatitis C respond to pegylated interferon-alpha/ribavirin combination therapy than to combination therapy with unpegylated interferon alpha. However, as with monotherapy, significant side effects develop during combination therapy, including hemolysis, flu-like symptoms, anemia, and fatigue. (Gary L. Davis. Gastroenterology 118:S104-S114, 2000).

Combination therapy with PEG-INTRON® (peginterferon alpha-2b) and REBETOL® (Ribavirin, USP) Capsules is available from Schering Corporation. REBETOL® (Schering Corporation) has also been approved in combination with INTRON® A (Interferon alpha-2b, recombinant, Schering Corporation). Roche's PEGASYS® (pegylated interferon alpha-2a) and COPEGUS® (ribavirin) are also approved for the treatment of HCV.

PCT Publication Nos. WO 99/59621, WO 00/37110, WO 01/81359, WO 02/32414 and WO 03/024461 by Schering Corporation disclose the use of pegylated interferon alpha and ribavirin combination therapy for the treatment of HCV. PCT Publication Nos. WO 99/15194, WO 99/64016, and WO 00/24355 by Hoffmann-La Roche Inc also disclose the use of pegylated interferon alpha and ribavirin combination therapy for the treatment of HCV.

(3) Substrate-based NS3 protease inhibitors (for example, Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (Llinas-Brunet et al, Hepatitis C inhibitor peptide analogues, PCT WO 99/07734).

(4) Non-substrate-based inhibitors, for example, 2,4,6-trihydroxy-3-nitro-benzamide derivatives (Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing a para-phenoxyphenyl group;

(5) Thiazolidine derivatives which show relevant inhibition in a reverse-phase HPLC assay with an NS3/4A fusion protein and NS5A/5B substrate (for example Sudo K. et al., Antiviral Research, 1996, 32, 9-18), especially compound RD-1-6250, possessing a fused cinnamoyl moiety substituted with a long alkyl chain, RD4 6205 and RD4 6193;

(6) Thiazolidines and benzanilides (for example Kakiuchi N. et al. J. EBS Letters 421, 217-220; and Takeshita N. et al. Analytical Biochemistry, 1997, 247, 242-246);

(7) A phenanthrenequinone possessing activity against protease in a SDS-PAGE and autoradiography assay isolated from the fermentation culture broth of Streptomyces sp., for example, Sch 68631 (Chu M. et al., Tetrahedron Letters, 1996, 37, 7229-7232), and Sch 351633, isolated from the fungus Penicillium griscofuluum, which demonstrates activity in a scintillation proximity assay (Chu M. et al., Bioorganic and Medicinal Chemistry Letters 9, 1949-1952);

(8) Selective NS3 inhibitors, for example, those based on the macromolecule elgin c, isolated from leech (Qasim M. A. et al., Biochemistry, 1997, 36, 1598-1607);

(9) Helicase inhibitors (for example Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554);

(10) Polymerase inhibitors for example nucleotide analogues, gliotoxin (Ferrari R. et al. Journal of Virology, 1999, 73, 1649-1654), and the natural product cerulenin (Lohmann V. et al., Virology, 1998, 249, 108-118);

(11) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 1999, 181, 251-257).

(12) Inhibitors of IRES-dependent translation (Ikeda N et al., Agent for the prevention and treatment of hepatitis C, Japanese Patent Pub. JP-08268890; Kai Y. et al. Prevention and treatment of viral diseases, Japanese Patent Pub. JP-10101591).

(13) Nuclease-resistant ribozymes (for example Maccjak, D. J. et al., Hepatology 1999, 30, abstract 995).

(14) Nucleoside analogs have also been developed for the treatment of Flaviviridae infections.

Idenix Pharmaceuticals, Ltd. discloses branched nucleosides, and their use in the treatment of HCV and flaviviruses and pestiviruses in US Patent Publication No. 2003/0050229 A1 and US Patent Publication No. 2003/0060400 A1, which correspond to International Publication Nos. WO 01/90121 and WO 01/92282. A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier.

Other patent applications disclosing the use of certain nucleoside analogs to treat hepatitis C virus include: International Patent Publication Nos. WO 01/32153 (PCT/CA00/01316; filed Nov. 3, 2000) and WO 01/60315 (PCT/CA01/00197; filed Feb. 19, 2001) filed by BioChem Pharma, Inc. (now Shire Biochem, Inc.); US Patent Publication No. 2002/0147160 and the corresponding International Patent Publication Nos. WO 02/057425 (PCT/US02/01531; filed Jan. 18, 2002) and WO 02/057287 (PCT/US02/03086; filed Jan. 18, 2002) filed by Merck & Co., Inc.; US Patent Publication Nos. 2003/083307 A1 and US 2003/008841 A1, and the corresponding International Patent Publication Nos. WO 02/18404 (PCT/EP01/09633; published Aug. 21, 2001); WO 02/100415 and WO 02/094289, filed by Hoffman-LaRoche; US Patent Publication No. 2003/028013 A1 and the corresponding International Patent Publication Nos. WO 03/062255 and WO 03/061385 filed by Ribapharm; and WO 01/79246 and WO 02/32920 filed by Pharmasset.

(15) Miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antioxidants (U.S. Pat. No. 5,922,757 to Chojkier et al.), squalene, amantadine, bile acids (U.S. Pat. No. 5,846,964 to Ozeki et al.), N-(phosphonoacetyl)-L-aspartic acid, (U.S. Pat. No. 5,830,905 to Diana et al.), benzenedicarboxamides (U.S. Pat. No. 5,633,388 to Diana et al.), polyadenylic acid derivatives (U.S. Pat. No. 5,496,546 to Wang et al.), 2′,3′-dideoxyinosine (U.S. Pat. No. 5,026,687 to Yarchoan et al.), and benzimidazoles (U.S. Pat. No. 5,891,874 to Colacino et al.).

(16) Other compounds currently in clinical development for treatment of hepatitis c virus include: Interleukin-10 by Schering-Plough, IP-501 by Interneuron, Merimebodib VX-497 by Vertex, AMANTADINE (Symmetrel) by Endo Labs Solvay, HEPTAZYME by RPI, IDN-6556 by Idun Pharma., XTL-002 by XTL., HCV/MF59 by Chiron, CIVACIR by NABI, LEVOVIRIN by ICN, VIRAMIDINE by ICN, ZADAXIN (thymosin alfa-1) by Sci Clone, CEPLENE (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

Combination and/or Alternation Therapies for the Treatment of Abnormal Cellular Proliferation

Examples of agents that have been identified as active against abnormal cellular proliferation, and thus can be used in combination or alternation with one or more nucleosides of general formula (I)-(V) or PALA include:

Alkylating Agents

Nitrogen Mustards: including, but not limited to Mechlorethamine (Hodgkin's disease, non-Hodgkin's lymphomas), Cyclophosphamide, Ifosfamide (acute and chronic lymphocytic leukemias, Hodgkin's disease, non-Hodgkin's lymphomas, multiple myeloma, neuroblastoma, breast, ovary, lung, Wilms' tumor, cervix, testis, soft-tissue sarcomas), Melphalan (L-sarcolysin) (multiple myeloma, breast, ovary), Chlorambucil (chronic lymphoctic leukemia, primary macroglobulinemia, Hodgkin's disease, non-Hodgkin's lymphomas).

Ethylenimines and Methylmelamines: including, but not limited to Hexamethylmelamine (ovary), Thiotepa (bladder, breast, ovary).

Alkyl Sulfonates: including, but not limited to Busulfan (chronic granuloytic leukemia).

Nitrosoureas: including, but not limited to Carmustine (BCNU) (Hodgkin's disease, non-Hodgkin's lymphomas, primary brain tumors, multiple myeloma, malignant melanoma), Lomustine (CCNU) (Hodgkin's disease, non-Hodgkin's lymphomas, primary brain tumors, small-cell lung), Semustine (methyl-CCNU) (primary brain tumors, stomach, colon), Streptozocin (STR) (malignant pancreatic insulinoma, malignant carcinoin).

Triazenes: including, but not limited to Dacarbazine (DTIC; dimethyltriazenoimidazole-carboxamide) (malignant melanoma, Hodgkin's disease, soft-tissue sarcomas).

Antimetabolites

Folic Acid Analogs: including, but not limited to Methotrexate (amethopterin) (acute lymphocytic leukemia, choriocarcinoma, mycosis fungoides, breast, head and neck, lung, osteogenic sarcoma).

Pyrimidine Analogs: including, but not limited to Fluorouracil (5-fluorouracil; 5-FU) Floxuridine (fluorodeoxyuridine; FUdR) (breast, colon, stomach, pancreas, ovary, head and neck, urinary bladder, premalignant skin lesions) (topical), Cytarabine (cytosine arabinoside) (acute granulocytic and acute lymphocytic leukemias).

Purine Analogs and Related Inhibitors: including, but not limited to Mercaptopurine (6-mercaptopurine; 6-MP) (acute lymphocytic, acute granulocytic and chronic granulocytic leukemia), Thioguanine (6-thioguanine: TG) (acute granulocytic, acute lymphocytic and chronic granulocytic leukemia), Pentostatin (2′-deoxycyoformycin) (hairy cell leukemia, mycosis fungoides, chronic lymphocytic leukemia).

Vinca Alkaloids: including, but not limited to Vinblastine (VLB) (Hodgkin's disease, non-Hodgkin's lymphomas, breast, testis), Vincristine (acute lymphocytic leukemia, neuroblastoma, Wilms' tumor, rhabdomyosarcoma, Hodgkin's disease, non-Hodgkin's lymphomas, small-cell lung).

Epipodophylotoxins: including, but not limited to Etoposide (testis, small-cell lung and other lung, breast, Hodgkin's disease, non-Hodgkin's lymphomas, acute granulocytic leukemia, Kaposi's sarcoma), Teniposide (testis, small-cell lung and other lung, breast, Hodgkin's disease, non-Hodgkin's lymphomas, acute granulocytic leukemia, Kaposi's sarcoma).

Natural Products

Antibiotics: including, but not limited to Dactinomycin (actinonmycin D) (choriocarcinoma, Wilms' tumor rhabdomyosarcoma, testis, Kaposi's sarcoma), Daunorubicin (daunomycin; rubidomycin) (acute granulocytic and acute lymphocytic leukemias), Doxorubicin (soft tissue, osteogenic, and other sarcomas; Hodgkin's disease, non-Hodgkin's lymphomas, acute leukemias, breast, genitourinary thyroid, lung, stomach, neuroblastoma), Bleomycin (testis, head and neck, skin and esophagus lung, and genitourinary tract, Hodgkin's disease, non-Hodgkin's lymphomas), Plicamycin (mithramycin) (testis, malignant hypercalcema), Mitomycin (mitomycin C) (stomach, cervix, colon, breast, pancreas, bladder, head and neck).

Enzymes: including, but not limited to L-Asparaginase (acute lymphocytic leukemia).

Biological Response Modifiers: including, but not limited to Interferon-alfa (hairy cell leukemia, Kaposi's sarcoma, melanoma, carcinoid, renal cell, ovary, bladder, non Hodgkin's lymphomas, mycosis fungoides, multiple myeloma, chronic granulocytic leukemia).

Miscellaneous Agents

Platinum Coordination Complexes: including, but not limited to Cisplatin (cis-DDP) Carboplatin (testis, ovary, bladder, head and neck, lung, thyroid, cervix, endometrium, neuroblastoma, osteogenic sarcoma).

Anthracenedione: including, but not limited to Mixtozantrone (acute granulocytic leukemia, breast).

Substituted Urea: including, but not limited to Hydroxyurea (chronic granulocytic leukemia, polycythemia vera, essential thrombocytosis, malignant melanoma).

Methylhydrazine Derivative: including, but not limited to Procarbazine (N-methylhydrazine, MIH) (Hodgkin's disease).

Adrenocortical Suppressant: including, but not limited to Mitotane (o,p′-DDD) (adrenal cortex), Aminoglutethimide (breast).

Adrenorticosteriods: including, but not limited to Prednisone (acute and chronic lymphocytic leukemias, non-Hodgkin's lymphomas, Hodgkin's disease, breast).

Progestins: including, but not limited to Hydroxprogesterone caproate, Medroxyprogesterone acetate, Megestrol acetate (endometrium, breast).

Antioangiogenesis Agents

Including, but not limited to Angiostatin, Endostatin.

Hormones and Antagonists

Estrogens: including, but not limited to Diethylstibestrol Ethinyl estradiol (breast, prostate)

Antiestrogen: including, but not limited to Tamoxifen (breast).

Androgens: including, but not limited to Testosterone propionate Fluxomyesterone (breast).

Antiandrogen: including, but not limited to Flutamide (prostate).

Gonadotropin-Releasing Hormone Analog: including, but not limited to Leuprolide (prostate).

Synthetic Protocol

Only carbocyclic nucleosides so far found in nature are adenine nucleosides, i.e., aristeromycin and neplanocins, and they are either extremely expensive or commercially not available. Thus, these types of nucleosides typically are chemically synthesized from scratch. The carbocylic derivative is prepared first and then the heterocyclic aglycon is constructed on the sugar to prepare carbocylic nucleosides or alternatively, the base is directly condensed with the carbocylic derivative, for example a purine base can be directly condensed with the carbocylic derivative.

Scheme 1 illustrates the synthesis of carbocyclic cytidine (227, Type I-a). The carbocylic intermediate 219 can be synthesized by any means known in the art. It is disclosed by Ali et al. (Tetrahedron Letters, 1990, 31, 1509) that D-ribonolactone 217 is converted into the pentanone intermediate 218. The ketone 218 can then be reduced by any known reducing agent, preferably sodium borohydride in methanol at 0° C. for one hour to afford alcohol 219. Sulfonylation of 219, preferably with mesyl chloride in methylene chloride in the presence of triethylamine at 0° C. for 2 hours gives 220, which is then treated with sodium azide in DMF at 140° C. overnight to give 221. The azide 221 can readily be reduced with any known reducing agent, e.g., Ph₃P (Staudinger procedure) or catalytic hydrogenolysis, preferably over palladium on carbon. The resulting amine 222 is subjected to Warrener-Shaw reaction with β-methoxyacryloylisocyanate in DMF, followed by ammonium hydroxide treatment to form protected carbocyclic uridine 224 via the linear intermediate 223. Conversion of uracil nucleoside 224 into protected carbocyclic cytidine (225) can be achieved by any means known in the art. The protecting groups of 225 are removed with acid, preferably with trifluoroacetic acid/water (2:1 v/v) at 50° C. for 3 hours, to give 226.

Sulfonylation of 219 with triflyl chloride in methylene chloride in the presence of triethylamine gives triflate, which, upon reaction with purine base, such as adenine, and sodium hydride in an inert solvent, such as acetonitrile or DMF directly affords the corresponding purine nucleoside (I-b type).

By using the same procedure but starting from L-ribonolactone, the corresponding L-nucleosides counterparts can be obtained.

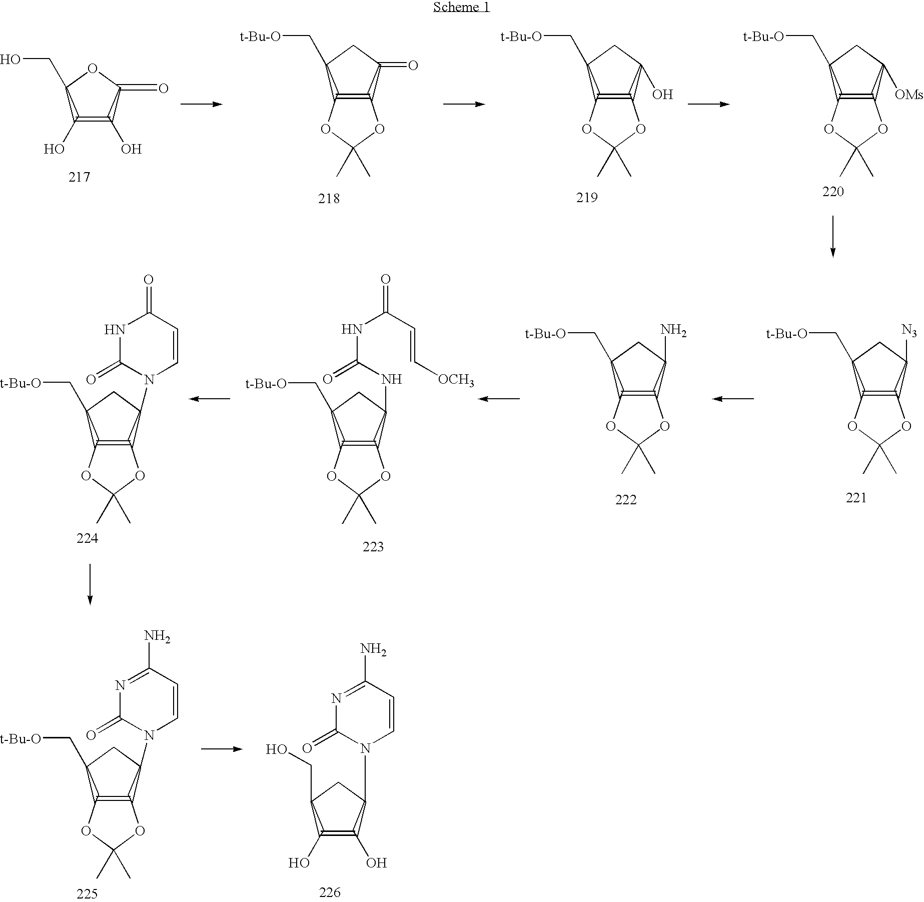

Alternatively, commercially available (1R)-(−)-azabicyclo[2.2.1]hept-5-en-3-one (228, Scheme 2) is converted into 2,3-dihydroxy-lactam 229 by osmium tetroxide oxidation. After methanolysis of 229 with methanolic hydrogen chloride, the product 230 is treated with 2,2-dimethoxypropane in acetone or 1,1-dimethoxycyclohexane in cyclohexanol to give a ketal, e.g., 231, which is reduced to 232 with sodium borohydride. The aminoalcohol 232 is converted into 2′,3′-O-cyclohexylidene-carbocyclic uridine by reaction with β-methoxyacryloylisocyanate, followed by ammonia treatment. Acid treatment, preferably with trifluoroacetic acid in methanol, gives carbocyclic uridine (233). Carbocyclic-5-fluorocytidine (227) can be obtained readily from 233 by the well-known means in the art.

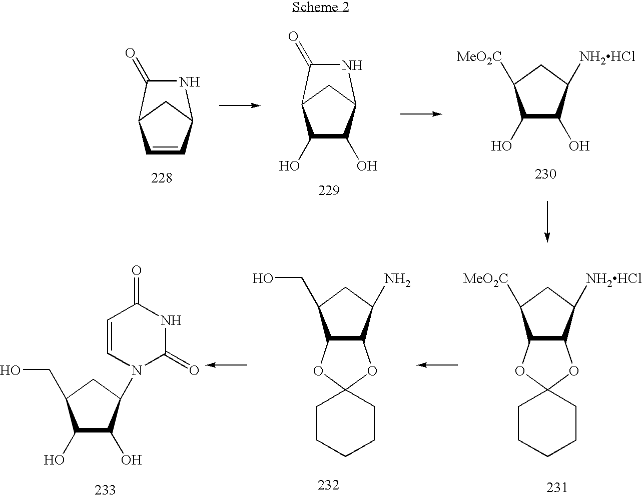

In a similar sequence of reactions but starting from the other optical isomer, (1R)-(+)-azabicyclo[2.2.1]hept-5-en-3-one, the corresponding L-nucleoside analogue can be obtained.

Scheme 3 shows the synthesis of 3,4-unsaturated carbocyclic nucleoside of type II Wolfe et al (J. Org. Chem., 1990, 55, 4712) prepared 261 from D-ribonolactone. Quenching the Michael addition of t-butoxymethyl group to (261, Scheme 3) with sulfinyl chloride, followed by heating the product with calcium carbonate gives cyclopentenone 262. Reduction of 262 with DIBAH followed by sulfonylation affords 263. Condensation of 263 (preferably R═CF₃) with purine base with NaH as described earlier gives purine nucleoside II-b, e.g., neplanocin A (264). Treatment of 263 (preferably R=Me) with NaN₃ gives 265 which can be readily converted into various pyrimidine nucleosides (II-a) including 266 by the procedure already described with Scheme 1.

Starting from L-ribonolactone, the corresponding L-nucleoside counterparts can be readily prepared.

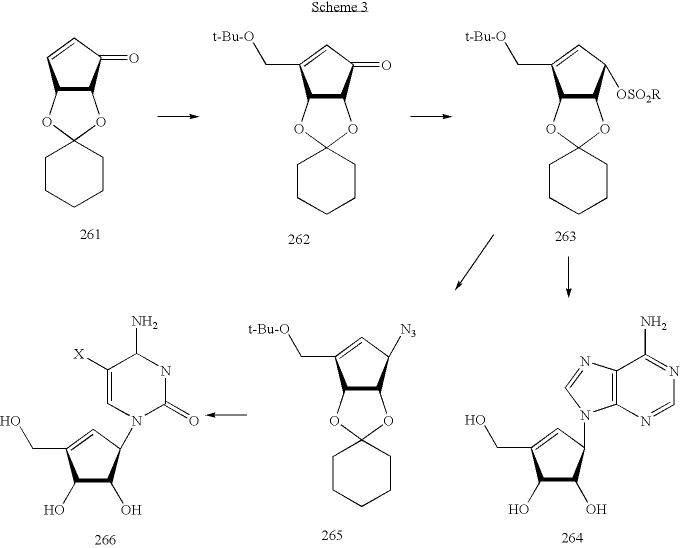

This invention is further illustrated in the following sections. The Experimental Details section and Examples contained therein are set forth to aid in an understanding of the invention. This section is not intended to, and should not be interpreted to, limit in any way the invention set forth in the claims that follow thereafter.

The following working examples provide a further understanding of the method of the present invention. These examples are of illustrative purposes, and are not meant to limit the scope of the invention. Equivalent, similar or suitable solvents, reagents or reaction conditions may be substituted for those particular solvents, reagents or reaction conditions described without departing from the general scope of the method.

### EXAMPLES

Melting points were determined in open capillary tubes on an Electrothermal digit melting point apparatus and are uncorrected. The UV absorption spectra were recorded on an Uvikon 931 (KONTRON) spectrophotometer in ethanol. ¹H-NMR spectra were run at room temperature with a Varian Unity Plus 400 spectrometer. Chemical shifts are given in ppm downfield from internal tetramethylsilane as reference. Deuterium exchange, decoupling experiments or 2D-COSY were performed in order to confirm proton assignments. Signal multiplicities are represented by s (singlet), d (doublet), dd (doublet of doublets), t (triplet), q (quadruplet), br (broad), m (multiplet). All J-values are in Hz. FAB mass spectra were recorded in the positive-(FAB>0) or negative-(FAB<0) ion mode on a JEOL DX 300 mass spectrometer The matrix was 3-nitrobenzyl alcohol (NBA) or a mixture (50:50, v/v) of glycerol and thioglycerol (GT). Specific rotations were measured on a Perkin-Elmer 241 spectropolarimeter (path length 1 cm) and are given in units of 10⁻¹ deg cm² g⁻¹. Elemental analyses were performed by Atlantic Microlab Inc. (Norcross, Ga.). Analyses indicated by the symbols of the elements or functions were within ±0.4% of theoretical values. Thin layer chromatography was performed on Whatman PK5F silica gel plates, visualization of products being accomplished by UV absorbency followed by charring with 10% ethanolic sulfuric acid and heating. Column chromatography was carried out on Silica Gel (Fisher, S733-1) at atmospheric pressure.

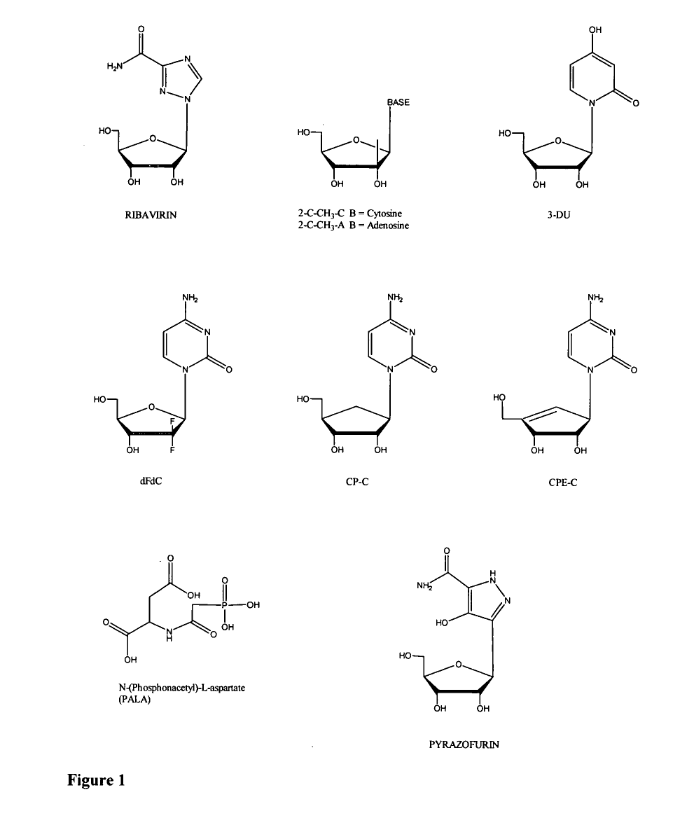

Chemicals and reagents. The following compounds were synthesized in the Pharmasset laboratories: tiazofurin, C2-MAD, guanazole, tezacytabine, 3-deazaurine (3DU), 6-aza-uridine, 2′-deoxy-5-fluorouridine, difluorodeoxycytidine (dFdC, gemcitabine), 2′-C-methyl-cytidine (2′-C—CH₃—C), and 2′-C-methyl-adenosine (2′-C—CH₃-A). PALA (NSC-224131), pyrazofurin (NSC-143095), and Brequinar (NSC-368390) were provided by the Drug Biosynthesis & Chemistry Branch, Developmental Therapeutics Program, Division of Cancer Treatment, National Cancer Institute (Bethesda, Md.). Cyclopentyl-cytosine (CP-C) and cyclopentenyl-cytosine (CPE-C) were synthesized by Dr. C. K. Chu (University of Georgia, Athens, Ga.) (FIG. 1). Mizoribine, methotrexate, 2-thio-6-azauridine, and deferoxamine mesylate were purchased from Sigma (Milwaukee, Wis.), mycophenolic acid (MPA) was kindly provided by Dr. Takashi Tsuji (Ajinomoto, Inc., Japan), and hydroxy urea was obtained from Dr. Raymond F. Schinazi (Emory University, Atlanta, Ga.). Ribavirin (1-β-D-ribofuranosyl-1,2,4-triazole-3-carboxyamide; Schering-Plough, Raritan, N.J.) and recombinant interferon alfa-2a (IFN-α-2a; Roferon-A, Hoffmann-La Roche Inc., NJ) served as controls in the replicon experiments.

### Example 1

HCV Replicon Tissue Culture

HCV-replicon RNA-containing Huh7 cells (Clone A cells; Apath, LLC, St. Louis, Mo.) were kept in exponential growth in DMEM media (high glucose, no pyruvate) containing 10% fetal bovine serum, 1× non-essential amino acids (100 units/ml), penicillin-streptomycin (100 μg/ml), glutamine (0.292 mg/ml), and G418 (1,000 μg/ml). Antiviral assays were performed in the same medium without G418. It was shown that the absence of G418 during antiviral testing has no effect on the levels of HCV-RNA (Stuyver, et al. “A ribonucleoside analogue that blocks the replication of bovine viral diarrhea and hepatitis C viruses in culture” Antimicrob. Agents Chemother., January 2003, 47 (1), 244-254). Cells were seeded in a 6-well plate at 10⁵ cells per well. Candidate antiviral compounds were tested as described (Stuyver, et al. “A ribonucleoside analogue that blocks the replication of bovine viral diarrhea and hepatitis C viruses in culture” Antimicrob. Agents Chemother., January 2003, 47 (1), 244-254). Incubation times differed according to the type of experiment. At the end of the incubation step, cells were counted using the trypan-blue exclusion method, and total cellular RNA was isolated (Rneasy 96 kit, Qiagen, CA). Replicon RNA and an internal control (TaqMan Ribosomal RNA Control Reagents, Applied Biosystems, CA) were amplified in a single-step, multiplex RT-PCR protocol, as recommended by the manufacturer and as described (Stuyver, et al. “A ribonucleoside analogue that blocks the replication of bovine viral diarrhea and hepatitis C viruses in culture” Antimicrob. Agents Chemother., January 2003, 47 (1), 244-254).

### Example 2

Growth of the Replicon Cells and Observation of HCV RNA Levels.

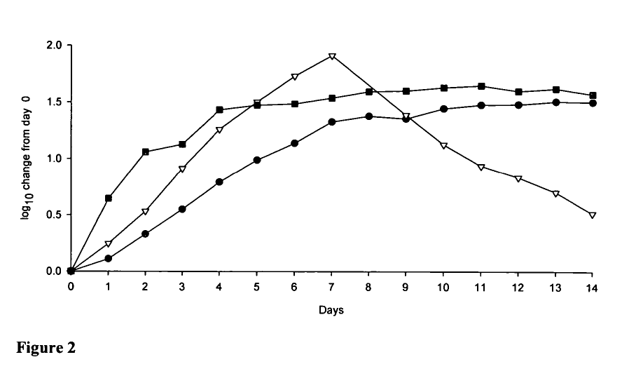

Evaluating candidate anti-HCV compounds in the replicon system is hampered by the fact that only cells in logarithmic growing conditions can be used. Cells that reach confluency—and hence enter into a G₀/G₁ cell cycle arrest—cannot maintain stable amounts of the replicon RNA levels per cell, as evidenced by a steady decrease in HCV RNA but not rRNA (FIG. 2) (Stuyver, et al. “A ribonucleoside analogue that blocks the replication of bovine viral diarrhea and hepatitis C viruses in culture” Antimicrob. Agents Chemother., January 2003, 47 (1), 244-254). This suggests that cellular factors that are required for replicon RNA replication and/or translation vary in abundance and become limited in resting cells. One of these factors might be the availability of sufficient levels of NTPs to support replicon synthesis.

Considerable incubation time-dependent fluctuations in the amounts of HCV RNA in replicon cells were previously observed (Pietschmann et al. “Characterization of cell lines carrying self-replicating hepatitis C virus RNAs” J. Virol., 2001, 75, 1252-1264). To further study these events in detail, a time course experiment was designed in which cell growth and HCV RNA dynamics in Huh7 cells were monitored over a 14-day period (Stuyver, et al. “A ribonucleoside analogue that blocks the replication of bovine viral diarrhea and hepatitis C viruses in culture” Antimicrob. Agents Chemother., January 2003, 47 (1), 244-254). During the first 7 days, the amount of HCV RNA in the culture increased over time more or less in parallel with the cell count and the intracellular rRNA levels (FIG. 2). This illustrates a steady state or small increase in HCV RNA copy number per cell. From day 8 onwards, cells reached a confluent monolayer, the rRNA levels did not significantly change from day 8 to day 14, but a sharp decrease in the amount of HCV RNA was then observed, indicating a significant drop in the HCV-RNA copy number per cell. These results illustrate that the HCV replicon RNA copy number is tightly coupled to the exponentially growing character of the host cell.

### Example 3

Control Experiments: Anti-HCV Effect of Previously Established Compounds in the HCV Replicon System.

Currently, IFN-α and ribavirin are the only approved drugs for treatment of HCV-infected patients. Besides these approved molecules, several others have been claimed to exert specific antiviral activity (Carroll, et al. “Inhibition of hepatitis C virus RNA replication by 2′-modified nucleoside analogs” J. Biol. Chem. 2003, 27, 27; Sommadossi, J. P., and P. Lacolla “Methods and compositions for treating hepatitis C virus” International Patent Application WO 01/190121, Idenix Pharmaceuticals; Walker, M. P., and Z. Hong “HCV RNA-dependent RNA polymerase as a target for antiviral development” Curr Opin Pharmacol, 2002, 2, 534-40).

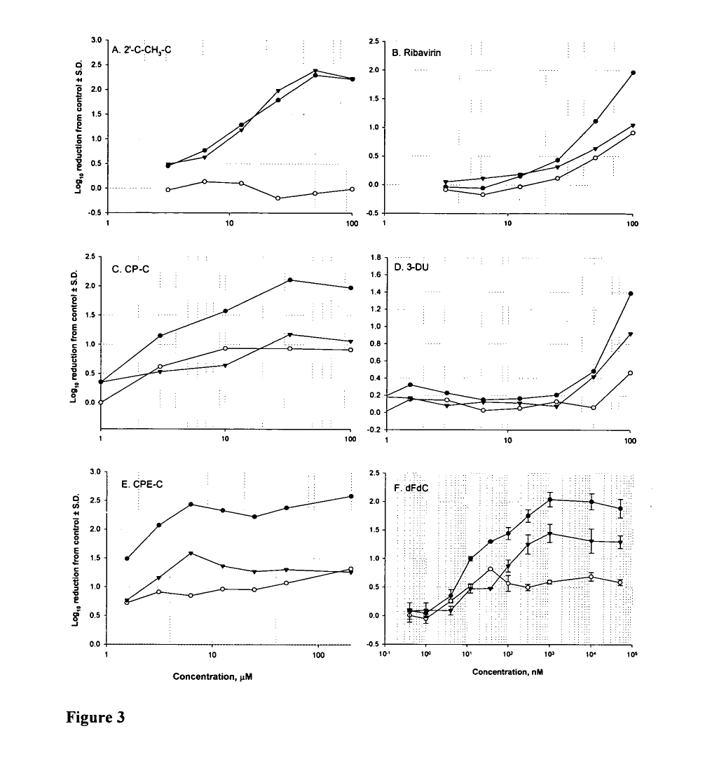

In a series of control experiments, IFN-α-2a, ribavirin, 2′-C—CH₃—C and 2′-C—CH₃-A were tested over a range of concentrations for their ability to reduce the HCV RNA levels in a dose-response manner in exponentially growing replicon cells after 4 days of compound exposure. When tested at 100 IU/ml, IFN-α-2a had only minimal effect on the rRNA levels (0.21±0.21 log₁₀ rRNA drop), and after correcting the log₁₀ drop for HCV RNA (1.57±0.26 log₁₀) for the observed rRNA reductions, a specific antiviral effect of 1.36±0.37 log₁₀ drop of HCV RNA was observed (Table 1). As previously published, IFN-α-2a showed a corrected EC₉₀ value of 4.5 IU/ml after 96 hr of incubation (Stuyver, et al. “A ribonucleoside analogue that blocks the replication of bovine viral diarrhea and hepatitis C viruses in culture” Antimicrob. Agents Chemother., January 2003, 47 (1), 244-254). Similar calculations were performed for the three other compounds (Table 1). EC₉₀ values for 2′-C—CH₃—C (FIG. 3a), ribavirin (FIG. 3b), and 2′-C—CH₃-A were found to be 10.4 μM, ˜100 μM, and <1 μM, respectively (Table 1; FIG. 3).

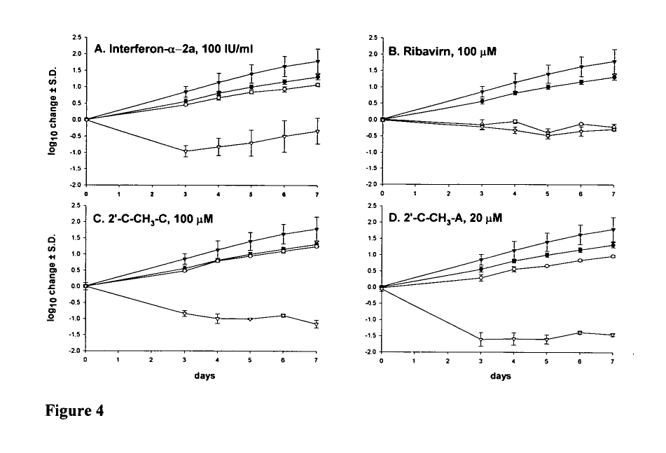

However, the EC₉₀ value at day 4 is a single static observation point, and does not provide information about cell growth dynamics or changes to the obligate requirement for logarithmic cell growth. Therefore, experiments were conducted to monitor HCV RNA levels and the dynamics of cell growth over a 7-day period. Based on the average of 6 experiments for IFN-α-2a, treated cells grew significantly slower (day 7: 1.07±0.06 log₁₀ increase from day 0) than the untreated cell controls (1.31±0.08 log₁₀ increase from day 0; p=0.003) (FIG. 4a). Although minor differences were observed in cell growth at day 4, they were found to be significant (control: 0.81±0.06; IFN-α-2a: 0.67±0.06; p=0.01). In addition, there was a significant drop in HCV RNA levels that was maintained over the 7-day period (control: 1.79±0.4; IFN-α-2a: −0.53±0.4; p=0.0005). Noteworthy here is the rebound of the viral RNA from day 4 onwards. 2′-C—CH₃—C (FIG. 4C) and 2′-C—CH₃-A (FIG. 4d) were found to be very potent in reducing the HCV RNA levels with, respectively, no (at 100 μM) and minimal—but significantly different—(at 20 μM) effects on cell proliferation (Table 1). Ribavirin was tested at 100 μM and found to cause a complete arrest in the cell proliferation (0.22±0.1 log₁₀ drop at day 7 compared to day 0; or 1.53 log₁₀ drop compared to the no treatment control on day 7) (FIG. 4b). Although there was a significant drop of 2.08 log₁₀ of HCV RNA levels on day 7 as compared to the no treatment controls, the ratio of HCV RNA copy number per cell in the treatment versus no-treatment control changed only marginally.

The control compound, 2′-C—CH₃—C, is a typical compound that does not inhibit the exponential growth of cells over the concentrations tested (FIG. 4c), does not affect rRNA levels, e.g. rRNA (FIG. 3a), but does reduce replicon HCV RNA levels significantly (corrected EC₉₀ at day 4=10.4 μM, Table 1). Thus, a specific antiviral effect on the HCV RNA replicon depends on at least some, if not a combination of all of the following conditions: (i) no effect on exponential cell growth, (ii) no or limited reduction in cellular host RNA levels, and (iii) reductions in the HCV RNA copy number per cell, as compared to the controls.

### Example 4

Antiviral Effect of Select Antimetabolites of the Present Invention.

Antimetabolites of the nucleotide biosynthesis pathways are known to prevent de novo synthesis of NTPs or dNTPs, resulting in either the slowing or stopping of cell division or in the death of the cells. Several classes of antimetabolites were evaluated in this study, including inhibitors for the IMPDH, RNR, CTPS, OOMPDC, ATC, and thymidylate synthase (TS) enzymes. These classes of inhibitors are known to directly change the intracellular pools of nucleotides (up-regulated because of blockage of the upstream pathway; or down-regulated because of blockage of the downstream pathway).

Replicon cells were incubated in the absence or presence of these anti-metabolites for 96 hours, after which intracellular rRNA and HCV RNA levels were quantified (Table 1).

Although several of these antimetabolites significantly lower the HCV RNA levels, an almost similar inhibitory effect was seen on the levels of rRNA (Table 1). After correction for cellular toxicity, the majority of these antimetabolites had no specific potential (corrected EC₉₀ values >100 μM) as anti-HCV agents.

However, compounds with known inhibitory effect on enzymes responsible for the de novo synthesis of UTP and CTP (aspartate transcarbamoylase (ATC, E.C.2.1.3.2); dihydro-orotate dehydrogenase (DHODH, E.C.3.5.2.3); orotidine 5′-monophosphate decarboxylase (OMPDC, E.C.4.1.1.23); CTP synthase (CTPS, E.C.6.3.4.2)) showed some antiviral effect. These inhibitors were tested in dose-response assays after 96 h of incubation, resulting in the following EC₉₀ values, corrected for rRNA reductions: CP-C=25 μM (FIG. 3C); 3-DU=˜100 μM (FIG. 3D); CPE-C=2.5 μM (FIG. 3E); pyrazofurin=3.8 μM; PALA=7.6 μM; and dFdC=0.17 μM (FIG. 3F). dFdC previously showed several antimetabolite activities, including inhibition of ribonucleotide reductase (RNR) and CTPS (Heinemann et al. “Gemcitabine: a modulator of intracellular nucleotide and deoxynucleotide metabolism” Semin Oncol. 1995, 22, 11-8; Plunkett et al. “Gemcitabine: metabolism, mechanisms of action, and self-potentiation” Semin Oncol., 1995, 22, 3-10).

### Example 5

Antiviral Effect of Inhibitors of the De Novo Synthesis of Ribo-Pyrimidines.

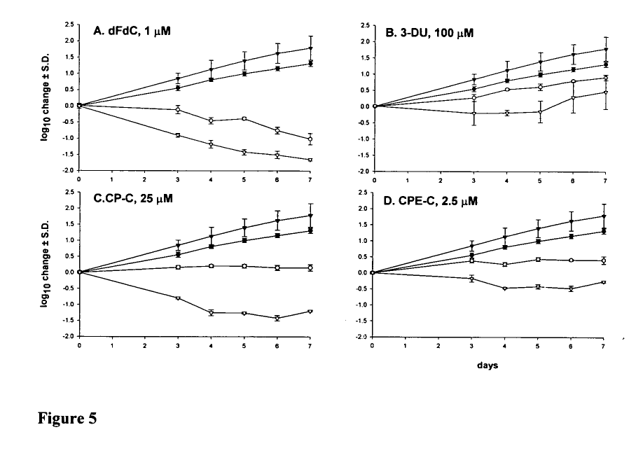

Selected inhibitors were evaluated for specific anti-HCV activity over a 7-day period (FIG. 5). The CTPS inhibitors caused cytostatic effects on the HCV replicon-containing Huh7 cell line when tested at their EC₉₀ values. Similar levels of cytostasis were also observed in the ribavirin experiment (FIG. 4b), although the inhibitors of the CTP and UTP de novo synthesis pathway seemed more specific in reducing HCV RNA levels than IMPDH inhibitors. The reduction of HCV RNA copies per cells was more prominent.

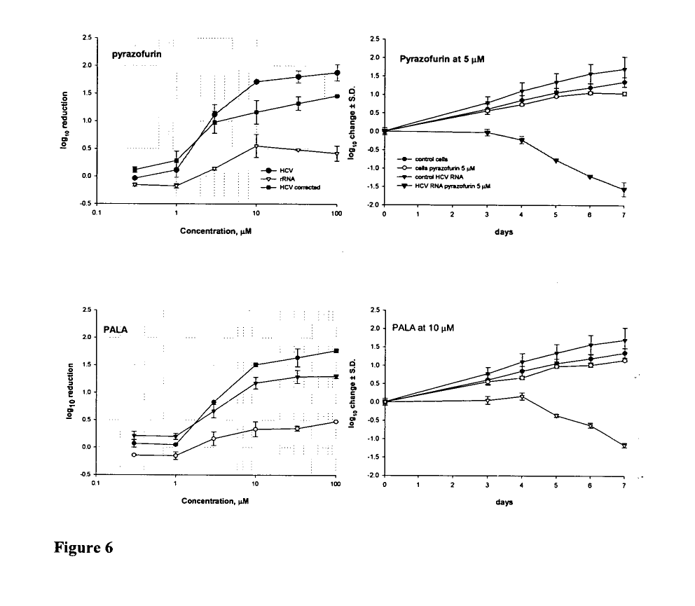

PALA and pyrazofurin showed very potent inhibition of the HCV RNA replication and there was minimal effect on cell growth over a seven-day assay, as compared to the no drug control (FIG. 6). In the latter assay, compounds were tested at their approximately EC₉₀ value for viral RNA reduction.

TS inhibitors block the conversion of dUMP to TMP, thereby reducing the available pool of TTP. Inhibitors of this type have been studied with regard to DNA viruses, such as Herpes and cytomegalovirues (Wachsman et al. “Anticytomegaloviral activity of methotrexate associated with preferential accumulation of drug by cytomegalovirus-infected cells” Antimicrob Agents Chemother., 1996, 40, 433-6), but little evidence is currently available that these TS inhibitors inhibit RNA viruses. As TTP is not a substrate for RNA polymerases (including the RdRP of HCV), this class of compounds can be seen as negative controls for the applied methodology. Although not tested in these studies, TS inhibitors can induce a cytotoxic or cytostatic outcome.

OMPDC is an enzyme that catalyzes the conversion of orotidine-5-phosphate to UMP; this is a crucial step in the biosynthesis of UTP. Treatment with certain inhibitors of this enzyme (e.g. 6-azauridine; 2-thio-6-azauridine) seemed to have little effect on cytoplasmic HCV RNA metabolism. Previously, 6-azauridine was found to be active against different flaviviruses (Crance et al. “Inhibition of sandfly fever Sicilian virus (Phlebovirus) replication in vitro by antiviral compounds” Res Virol. 1997, 148, 353-65; Morrey et al. “Identification of active antiviral compounds against a New York isolate of West Nile virus” Antiviral Res. 2002, 55, 107-16; Neyts et al. “Use of the yellow fever virus vaccine strain 17D for the study of strategies for the treatment of yellow fever virus infections” Antiviral Res. 1996, 30, 125-32), and the lack of any antiviral effect in the HCV RNA replicon system remains unexplained. It cannot be excluded that either salvage pyrimidine pathways combined with uptake of uracil or uridine from the culture media compensate for the inhibition. For 6-azauridine, the replicon experiments were repeated using dialyzed fetal calf serum in the medium, but essentially the same results were obtained.

Pyrazofurin, however, showed antiviral activity, as the molecule has been shown to possess against some viruses previously (Neyts et al. “Use of the yellow fever virus vaccine strain 17D for the study of strategies for the treatment of yellow fever virus infections” Antiviral Res. 1996, 30, 125-32; De Clercq et al. “Broad-spectrum antiviral and cytocidal activity of cyclopentenylcytosine, a carbocyclic nucleoside targeted at CTP synthetase” Biochem Pharmacol., 1991, 41, 1821-9). Pyrazofurin, in addition to inhibiting OMPDC has been reported to inhibit DHODH (Balzarini et al. “Effect of antimetabolite drugs of nucleotide metabolism on the anti-human immunodeficiency virus activity of nucleoside reverse transcriptase inhibitors” Pharmacol Ther. 2000, 87, 175-87). It is possible that the inhibition of OMPDC is compensated for by other cellular salvage pathways, and hence, inhibition at this level does not result in any specific antiviral effect, while inhibition of DHODH by essentially the same inhibitor can not be compensated for, and consequently results in the observed antiviral effect. The biological activity of pyrazofurin and 6-azauridine is at the level of monophosphate (Suttle, D. P., and G. R. Stark “Coordinate overproduction of orotate phosphoribosyltransferase and orotidine-5′-phosphate decarboxylase in hamster cells resistant to pyrazofurin and 6-azauridine” J. Biol. Chem. 1979, 254, 4602-7).

Certain IMPDH inhibitors inhibit the key enzyme step in purine nucleotide biosynthesis. Although several compounds belonging to this class were previously shown to be potent inhibitors in active virus production (Markland et al. 2000. Broad-spectrum antiviral activity of the IMP dehydrogenase inhibitor VX-497: a comparison with ribavirin and demonstration of antiviral additivity with alpha interferon. Antimicrob Agents Chemother. 44:859-866; Stuyver, et al. “Inhibitors of the IMPDH enzyme as potential anti-bovine viral diarrhea virus agents” Antiviral Chem Chemother. 2003, 13, 49-56), little specificity is observed when evaluated on the HCV replicon.

Certain CTPS inhibitors were shown to have potential against the HCV replicon, with CPE-C being the most potent. These compounds showed antiviral effects, and antiproliferative effects against a wide variety of human and murine tumor lines, including a panel of human gliosarcoma and astrocytoma lines (Agbaria et al. 1997. Antiproliferative effects of cyclopentenyl cytosine (NSC 375575) in human glioblastoma cells Oncol Res. 9:111-8; De Clercq et al. 1991. Broad-spectrum antiviral and cytocidal activity of cyclopentenylcytosine, a carbocyclic nucleoside targeted at CTP synthetase Biochem Pharmacol. 41:1821-9; Politi et al. 1995. Phase I clinical trial of continuous infusion cyclopentenyl cytosine. Cancer Chemother Pharmacol. 36:513-23). This effect is produced primarily by the 5′-triphosphate metabolite (e.g. CPEC-TP). Dose-dependent accumulation of CPEC-TP was accompanied by a concomitant decrease in CTP pools, with 50% depletion of the latter being achieved at a CPE-C level of about 0.1 μM.

dFdC was originally investigated for its antiviral effects (Bianchi et al., 1994. Inhibition of ribonucleotide reductase by 2′-substituted deoxycytidine analogs: possible application in AIDS treatment Proc Natl Acad Sci USA. 91:8403-7), but has since been developed as an antineoplastic agent. dFdC is a cell cycle-specific agent that primarily targets cells undergoing DNA synthesis (S-phase). The actions of dFdC can be summarized as follows: (i) dFdC-DP inhibits RNR, resulting in reduced concentrations of dCTP; (ii) reduced levels of dCTP result in a favorable incorportation of dFdC-TP into DNA, resulting in DNA strand breaks and cell death; (iii) reduced cellular dCTP levels result in an increased activity of deoxycytidine kinase, causing self-potentiation of dFdC; (iv) dFdC-TP inhibits dCMP deaminase; and finally, (v) high concentrations of dFdC-TP inhibits CTPS (Heinemann et al. 1995. Gemcitabine: a modulator of intracellular nucleotide and deoxynucleotide metabolism Semin Oncol. 22:11-8; Plunkett et al. 1995. Gemcitabine: metabolism, mechanisms of action, and self-potentiation Semin Oncol. 22:3-10). As none of the other RNR inhibitors tested (HU, tezacytabine, deferoxamine, guanazole) showed any specific inhibition of the replicon, the antiviral effect of dFdC may be ascribed to the CTPS inhibition. This would fit with the hypothesis that reducing the levels of UTP and/or CTP by any type of inhibitor (PALA, pyrazofurin, CP-C, CPE-C, and dFdC) could result in an antiviral effect.

Although almost all compounds tested induced cytostasis, not all antimetabolites had the ability to reduce the HCV RNA replicon copy number per cell. Typically, IMPDH inhibitors only showed minimal reduction, while CTPS inhibitors were more potent. Thus, the intracellular nucleotide pools play an important role in maintaining the steady-state levels of HCV RNA copy number. When cells enter drug induced-cytostasis, reductions in CTP levels (or pyrimidines more generally) seem to have a bigger impact than the levels of GTP (or purines more generally) on HCV RNA turnover.

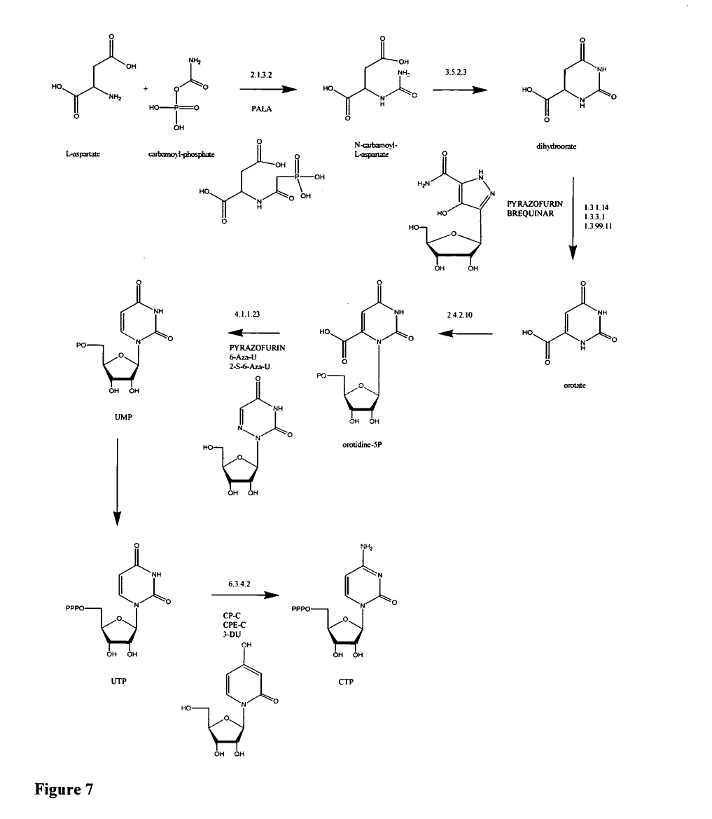

Replicon RNA turnover is an equilibrium between active production through RdRP versus HCV replicon RNA half-life. Exponentially growing cells are primarily dependent on de novo NTP synthesis, whereas confluent cells more often use salvage pathways to support their NTP needs. This suggests that certain antimetabolites (de novo pyrimidine nucleoside inhibitors) may have the capacity to mimic the observation seen in confluent cells, namely a rapid degradation of the replicon RNA pool under cytostatic conditions. The de novo synthesis of pyrimidines could be important, and inhibiting any of the synthetic steps may result in measurable reduction of viral RNA. An overview of the synthetic pathway and the known inhibitors is given in FIG. 7.

If limited availability of intracellular CTP is responsible for the destruction of replicon RNA steady state in confluent, untreated cells (as shown in FIG. 2), then the observation seen with CTPS inhibitors may be interpreted as a non-specific antiviral effect.

TABLE 1

| | | | Corrected | Corrected | |
|---|---|---|---|---|---|
| | Log₁₀ RNA | | HCV RNA | HCV RNA | Corrected |
| | reduction¹ (100 μM) | | log₁₀ reduction¹ | log₁₀ reduction | HCV RNA |
| Compound | HCV | rRNA | at 100 μM | at 10 μM | EC₉₀ (μM) |

IMPDH inhibitors (E.C. 1.1.1.205)

| | | | | | |
|---|---|---|---|---|---|
| Ribavirin | 1.96 ± 0.28 | 0.91 ± 0.12 | 1.05 ± 0.29 | 0.16 ± 0.10 | ˜100 |
| Mizoribine | 0.29 ± 0.74 | 0.21 ± 0.50 | 0.08 ± 0.82 | −0.14 ± 0.12 | >100 |
| Tiazofurin | 0.86 ± 0.27 | 0.99 ± 0.35 | −0.13 ± 0.37 | 0.04 ± 0.10 | >100 |
| MPA | 1.15 ± 0.43 | 1.09 ± 0.28 | 0.07 ± 0.47 | 0.22 ± 0.01 | >100 |
| C2-MAD | 1.09 ± 0.21 | 1.00 ± 0.15 | 0.08 ± 0.24 | 0.36 ± 0.21 | >100 |

Ribonucleotide reductase inhibitors (E.C. 1.17.4.1; E.C. 1.17.4.2)

| | | | | | |
|---|---|---|---|---|---|
| Guanazole | 0.25 ± 0.11 | 0.07 ± 0.03 | 0.32 ± 0.08 | 0.05 ± 0.08 | >100 |
| Hydroxy Urea | 0.17 ± 0.08 | 0.25 ± 0.20 | −0.08 ± 0.16 | 0.06 ± 0.04 | >100 |
| Tezacytabine | 1.59 ± 0.08 | 1.78 ± 0.69 | −0.19 ± 0.49 | 0.63 ± 0.07 | >100 |
| Deferoxamine | 1.00 ± 0.06 | 0.92 ± 0.08 | 0.08 ± 0.03 | 0.17 ± 0.11 | >100 |

CTP synthase inhibitors (E.C. 6.3.4.2.)

| | | | | | |
|---|---|---|---|---|---|
| dFdC | 1.87 ± 0.16 | 0.59 ± 0.05 | 1.29 ± 0.11 | 1.32 ± 0.08 | 0.17 |
| CP-C | 1.97 ± 0.38 | 0.91 ± 0.13 | 1.06 ± 0.26 | 0.64 ± 0.10 | 25 |
| CPE-C | 2.47 ± 0.33 | 1.21 ± 0.16 | 1.26 ± 0.51 | 1.43 ± 0.01 | 2.5 |
| 3DU | 1.41 ± 0.09 | 0.48 ± 0.11 | 0.94 ± 0.20 | 0.13 ± 0.10 | ˜100 |

Orotidine-MP decarboxylase (E.C. 4.1.1.23)

| | | | | | |
|---|---|---|---|---|---|
| 6-aza uridine | 0.25 ± 0.09 | 0.61 ± 0.18 | −0.36 ± 0.16 | 0.12 ± 0.05 | >100 |
| 2-thio-6-azauridine | 0.16 ± 0.04 | −0.02 ± 0.12 | 0.19 ± 0.09 | 0.12 ± 0.10 | >100 |
| pyrazofurin | 1.88 ± 0.05 | 0.42 ± 0.03 | 1.46 ± 0.08 | 1.16 ± 0.21 | 3.80 |

Aspartate transcarbamoylase (E.C. 2.1.3.2)

| | | | | | |
|---|---|---|---|---|---|
| PALA | 1.77 ± 0.02 | 0.48 ± 0.02 | 1.30 ± 0.05 | 1.18 ± 0.11 | 7.60 |

Thymidylate synthase inhibitors (E.C. 2.1.1.45)

| | | | | | |
|---|---|---|---|---|---|
| 2′-deoxy-5FU | 0.76 ± 0.06 | 0.73 ± 0.35 | 0.04 ± 0.25 | 0.23 ± 0.05 | >100 |
| Methotrexate | 0.18 ± 0.01 | 0.07 ± 0.10 | 0.11 ± 0.09 | 0.15 ± 0.01 | >100 |

Controls

| | | | | | |
|---|---|---|---|---|---|
| Interferon | 1.57 ± 0.26 | 0.21 ± 0.21 | 1.36 ± 0.37 | NA | 4.5 IU/ml |
| 2′C—CH3-A | 2.32 ± 0.11 | 2.96 ± 0.08 | −0.64 ± 0.18 | 2.05 | <1 |
| 2′C—CH3—C | 2.20 ± 0.52 | −0.02 ± 0.05 | 2.21 ± 0.47 | 1.0 | 10.4 |

¹IFN tested at 100 IU/ml; dFdC tested at 50 μM

### Example 6

Reversal Studies.

A series of experiments was then conducted to study the possibility of preventing the observed antiviral and cytostatic effects. Cells were incubated with the test compound and, simultaneously, the natural ribo- or 2′-deoxy nucleosides (adenosine, guanosine (G), cytidine (C), uridine (U), 2′-deoxycytidine (dC), 2′-deoxyuridine, thymidine, 2′-deoxyguanosine (dG), and 2′-deoxyadenosine) (Table 2).

The antiviral effect of the known antiviral compounds, IFN-α-2a and 2′-C—CH3-A, could not be prevented by any of the natural nucleosides. As expected for IMPDH inhibitors, the effect of ribavirin on cell growth and HCV replicon RNA replication was prevented by dG and G. In the case of dFdC, observed toxicities and antiviral effects were prevented by dC. In line with expectations for CTPS inhibitors, addition of cytidine to the culture medium compensated for the inhibitory effects. Surprisingly, when CPE-C was tested at lower concentrations (1 μM), the antimetabolite effects could be partially prevented by 50 μM of uridine in the media (Table 2). The effects of the inhibitors of the ATC, DHODH, and OMPDC enzymes could be prevented by addition of uridine to the culture media.

The invention has been described with reference to various specific and preferred embodiments and techniques. However, it should be understood that many variations and modifications will be obvious to those skilled in the art from the foregoing detailed description of the invention and may be made while remaining within the spirit and scope of the invention.

TABLE 2

| | | log₁₀ reduction¹ | | | log₁₀ reduction² | |
|---|---|---|---|---|---|---|
| Compound | Concentration, μM | HCV | rRNA | prevented by | HCV | rRNA |
| IFN | 3125 IU/ml | 1.62 ± 0.05 | 0.26 ± 0.18 | — | NA | NA |
| ribavirin | 100 | 1.96 ± 0.28 | 0.91 ± 0.12 | G | 0.43 ± 0.06 | 0.22 ± 0.10 |
| | | | | dG | 0.40 ± 00.01 | 0.07 ± 0.16 |
| 2′-C—CH3—C | 25 | 1.62 ± 0.05 | −0.01 ± 0.02 | C | 0.48 ± 0.02 | 0.14 ± 0.04 |
| 2′-C—CH3-A | 100 | 2.32 ± 0.11 | 2.96 ± 0.08 | — | NA | NA |
| dFdC | 1 | 1.89 ± 0.07 | 0.52 ± 0.03 | dC | 0.06 ± 0.00 | 0.07 ± 0.02 |
| CP-C | 20 | 1.80 ± 0.07 | 0.87 ± 0.08 | C | 0.11 ± 0.01 | 0.02 ± 0.00 |
| | 100 | 2.32 ± 0.08 | 1.21 ± 0.02 | C | 0.32 ± 0.11 | 0.17 ± 0.01 |
| CPE-C | 2 | 1.76 ± 0.04 | 0.99 ± 0.04 | C | 0.30 ± 0.06 | −0.04 ± 0.01 |
| | | | | U | 0.58 ± 0.04 | 0.32 ± 0.03 |
| 3DU | 100 | 1.41 ± 0.09 | 0.48 ± 0.11 | C | 0.35 ± 0.03 | 0.13 ± 0.03 |
| | | | | U | 0.37 ± 0.03 | 0.22 ± 0.07 |
| pyrazofurin | 100 | 1.88 ± 0.05 | 0.42 ± 0.03 | U | 0.35 ± 0.03 | −0.01 ± 0.04 |
| PALA | 100 | 1.77 ± 0.02 | 0.48 ± 0.02 | U | 0.14 ± 0.03 | −0.13 ± 0.10 |

NA: not applicable
¹log₁₀ RNA reduction at given concentration;
²log₁₀ RNA reduction at given concentration including the natural nucleoside at 50 μM that is preventing the antiviral and toxic effect.

1. A method for the treatment and/or prophylaxis of a Flaviviridae infection or disease associated with abnormal cellular proliferation in a host in need thereof, comprising administering to said host an effective treatment amount of a β-D-nucleoside of the general formula (IV-a*): 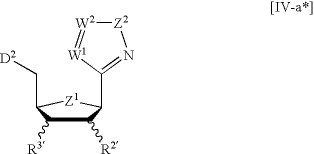
or a pharmaceutically acceptable salt and/or prodrug thereof, wherein:
each D² is independently OH, SH, NH₂, NHR⁴, or OD, wherein D is hydrogen, alkyl, acyl, monophosphate, diphosphate, triphosphate, monophosphate ester, diphosphate ester, triphosphate ester, phospholipid or amino acid;
each Z¹ is independently O, S, CH₂, CF₂, C(═O), or C(═CH₂);
each Z² is independently O, S, Se, C(═O), C(═S), C(═CH₂), NH, or NR⁵;
each W¹ and W² is independently N or CR^{1′};
each R^{1′} is independently hydrogen, F, Cl, Br, 1, CH₃, CH₂CH₃, Pr, i-Pr, n-Bu, i-Bu, t-Bu, CH₂CN, CH₂CO₂CH₃, CH₂C(═O)NH₂, CH₂C(═S)NH₂, C(═O)NH₂, C(═S)NH₂, C(═NH)NH₂, C(═O)NHOH, C(═O)NHNH₂, CH₂NH₃, NH₂, NHCH₃, N(CH₃)₂, NHCH₂CH₃, OH, OCH₃, OCH₂CH₃, SH, SCH₃, SCH₂CH₃, CO₂H, CN, or CHR*NH₂;
each R* is hydrogen, F, Cl, Br, or I;
each R^{2′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R^{3′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl;
each R⁵ is independently hydrogen, CH₃, CH₂CH₃, Pr, i-Pr, n-Bu, i-Bu, t-Bu, CH₂CN, CH₂CO₂CH₃, CH₂C(═O)NH₂, CH₂C(═S)NH₂, C(═O)NH₂, or C(═S)NH₂; and
such that there are no more than three ring-heteroatoms;
optionally in a pharmaceutically acceptable carrier or diluent.

2. The method of claim 1, wherein Z¹ is O.

3. The method of claim 1, wherein Z¹ is S.

4. The method of claim 1, wherein Z¹ is CH₂.

5. The method of claim 1, wherein Z¹ is CF₂.

6. A method for the treatment and/or prophylaxis of a Flaviviridae infection or disease associated with abnormal cellular proliferation in a host in need thereof, comprising administering to said host an effective treatment amount of a β-D-nucleoside of the general formula (IV-b*): 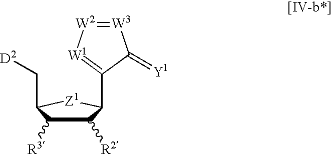
or a pharmaceutically acceptable salt and/or prodrug thereof, wherein:
each D² is independently OH, SH, NH₂, NHR⁴, or OD, wherein D is hydrogen, alkyl, acyl, monophosphate, diphosphate, triphosphate, monophosphate ester, diphosphate ester, triphosphate ester, phospholipid or amino acid;
each Z¹ is independently O, S, CH₂, CF₂, C(═O), or C(═CH₂);
each Y¹ is independently O, S, Se, or NH;
each W¹ and W² is independently N or CR^{1′};
each W³ is independently N, CH, CCH₃, CF, CCl, CBr, Cl, CCO₂H, CCO₂CH₃, CCONH₂, CC(═S)NH₂, or CCN;
each R^{1′} is independently hydrogen, halogen (F, Cl, Br or I), CH₃ (Me), CH₂CH₃ (Et), Pr, i-Pr, n-Bu, i-Bu, t-Bu, CH₂CN, CH₂CO₂CH₃, CH₂C(═O)NH₂, CH₂C(═S)NH₂, C(═O)NH₂, C(═S)NH₂, NH₂, NHCH₃, N(CH₃)₂, NHCH₂CH₃, OH, OCH₃, OCH₂CH₃, SH, SCH₃, SCH₂CH₃, CO₂H, or CN;
each R^{2′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R^{3′} independently is hydrogen, F, Cl, Br, I, CH₃, CH₂OH, CH₂F, CH₂SH, CH₂SCH₃, CH₂N₃, CH₂NH₂, OH, OCH₃, or NH₂; and
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl;
optionally in a pharmaceutically acceptable carrier or diluent.

7. The method of claim 6, wherein Z¹ is O.

8. The method of claim 6, wherein Z¹ is S.

9. The method of claim 6, wherein Z¹ is CH₂.

10. The method of claim 6, wherein Z¹ is CF₂.

11. A method for the treatment and/or prophylaxis of a Flaviviridae infection or disease associated with abnormal cellular proliferation in a host in need thereof, comprising administering to said host an effective treatment amount of a p-D-nucleoside of the general formula (IV-c*): 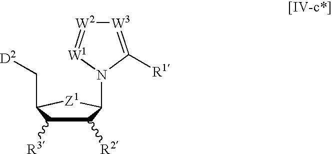
or a pharmaceutically acceptable salt and/or prodrug thereof, wherein:
each D² is independently OH, SH, NH₂, NHR⁴, or OD, wherein D is hydrogen, alkyl, acyl, monophosphate, diphosphate, triphosphate, monophosphate ester, diphosphate ester, triphosphate ester, phospholipid or amino acid;
each Z¹ is independently O, S, CH₂, CF₂, C(═O), or C(═CH₂);
each Y¹ is independently O, S, Se, or NH;
each W¹, W², and W³ is independently N or CR^{1′};
each R^{1′} is independently hydrogen, F, Cl, Br, I, CH₃, CH₂CH₃, Pr, i-Pr, n-Bu, i-Bu, t-Bu, CH₂CN, CH₂CO₂CH₃, CH₂C(═O)NH₂, CH₂C(═S)NH₂, C(═O)NH₂, C(═S)NH₂, NH₂, NHCH₃, N(CH₃)₂, NHCH₂CH₃, OH, OCH₃, OCH₂CH₃, SH, SCH₃, SCH₂CH₃, CO₂H, or CN;
each R^{2′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R^{3′} independently is hydrogen, F, Cl, Br, I, CH₃, CH₂OH, CH₂F, CH₂SH, CH₂SCH₃, CH₂N₃, CH₂NH₂, OH, OCH₃, or NH₂; and
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl;
optionally in a pharmaceutically acceptable carrier or diluent.

12. The method of claim 11, wherein Z¹ is O.

13. The method of claim 11, wherein Z¹ is S.

14. The method of claim 11, wherein Z¹ is CH₂.

15. The method of claim 11, wherein Z¹ is CF₂.

16. A method for the treatment and/or prophylaxis of a Flaviviridae infection or disease associated with abnormal cellular proliferation in a host in need thereof, comprising administering to said host an effective treatment amount of a β-D-nucleoside of the general formula (IV-d*): 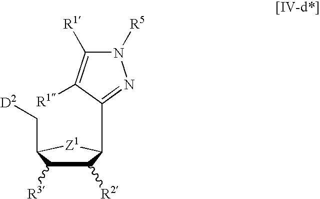
or a pharmaceutically acceptable salt and/or prodrug thereof, wherein:
each D² is independently OH, SH, NH₂, NHR⁴, or OD, wherein D is hydrogen, alkyl, acyl, monophosphate, diphosphate, triphosphate, monophosphate ester, diphosphate ester, triphosphate ester, phospholipid or amino acid;
each Z¹ is independently O, S, CH₂, CF₂, C(═O), or C(═CH₂);
each R^{1′} is independently CN, CO₂CH₃, C(═O)NH₂, C(═S)NH₂, or C(═NH)NH₂;
each R^{1″} is independently OH, SH, NH₂, or NHR⁵;
each R^{2′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R^{3′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl; and
each R⁵ is independently is hydrogen, optionally substituted or unsubstituted lower alkyl, or an optionally substituted or unsubstituted acyl;
optionally in a pharmaceutically acceptable carrier or diluent.

17. The method of claim 16, wherein Z¹ is O.

18. The method of claim 16, wherein Z¹ is S.

19. The method of claim 16, wherein Z¹ is CH₂.

20. The method of claim 16, wherein Z¹ is CF₂.

21. A method for the treatment and/or prophylaxis of a Flaviviridae infection or disease associated with abnormal cellular proliferation in a host in need thereof, comprising administering to said host an effective treatment amount of a β-D-nucleoside of the formula: 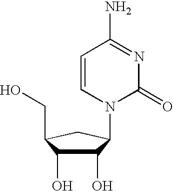
or a pharmaceutically acceptable salt and/or prodrug thereof, optionally in a pharmaceutically acceptable carrier or diluent.

22. A method for the treatment and/or prophylaxis of a Flaviviridae infection or disease associated with abnormal cellular proliferation in a host in need thereof, comprising administering to said host an effective treatment amount of a β-D-nucleoside of the formula: 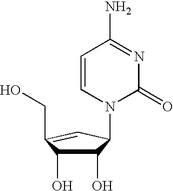
or a pharmaceutically acceptable salt and/or prodrug thereof, optionally in a pharmaceutically acceptable carrier or diluent.

23. A method for the treatment and/or prophylaxis of a Flaviviridae infection or disease associated with abnormal cellular proliferation in a host in need thereof, comprising administering to said host an effective treatment amount of a β-D-nucleoside of the formula: 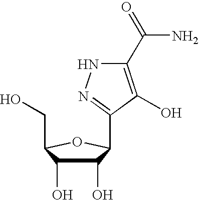
or a pharmaceutically acceptable salt and/or prodrug thereof, optionally in a pharmaceutically acceptable carrier or diluent.

24. A method for the treatment and/or prophylaxis of a Flaviviridae infection or disease associated with abnormal cellular proliferation in a host in need thereof, comprising administering to said host an effective treatment amount of a β-D-nucleoside of the formula: 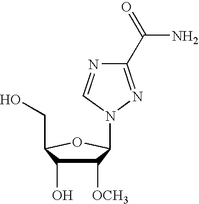
or a pharmaceutically acceptable salt and/or prodrug thereof, optionally in a pharmaceutically acceptable carrier or diluent.

25. A method for the treatment and/or prophylaxis of a Flaviviridae infection or disease associated with abnormal cellular proliferation in a host in need thereof, comprising administering to said host an effective treatment amount of N-(phosphonoacetyl)-L-aspartate (PALA), or its pharmaceutically acceptable salt and/or prodrug, optionally in a pharmaceutically acceptable carrier or diluent.

26. The method of claim 1, wherein the pharmaceutically acceptable carrier is suitable for oral delivery.

27. The method of claim 1, wherein the pharmaceutically acceptable carrier is suitable for intravenous delivery.

28. The method of claim 1, wherein the pharmaceutically acceptable carrier is suitable for parenteral delivery.

29. The method of claim 1, wherein the pharmaceutically acceptable carrier is suitable for intradermal delivery.

30. The method of claim 1, wherein the pharmaceutically acceptable carrier is suitable for subcutaneous delivery.

31. The method of claim 1, wherein the pharmaceutically acceptable carrier is suitable for topical delivery.

32. The method of claim 1, wherein the effective compound is in the form of a dosage unit, such that said dosage unit contains 10 to 1500 mg of the compound.

33. The method of claim 1, wherein the effective compound is in the form of a dosage unit that is a tablet or capsule.

34. The method of claim 1, wherein the host is a human.

35. The method of claim 1, wherein the Flaviviridae infection is an HCV infection.

36. The method of claim 35, wherein the host is a human.

37. A method for the treatment and/or prophylaxis of an HCV infection in a host in need thereof, comprising administering to said host an effective treatment amount of a β-D-nucleoside of the general formula (IV-a*): 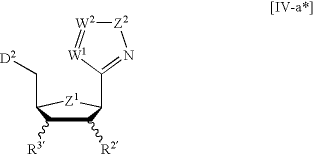
or a pharmaceutically acceptable salt and/or prodrug thereof, wherein:
each D² is independently OH, SH, NH₂, NHR⁴, or OD, wherein D is hydrogen, alkyl, acyl, monophosphate, diphosphate, triphosphate, monophosphate ester, diphosphate ester, triphosphate ester, phospholipid or amino acid;
each Z¹ is independently O, S, CH₂, CF₂, C(═O), or C(═CH₂);
each Z² is independently O, S, Se, C(═O), C(═S), C(═CH₂), NH, or NR⁵;
each W¹ and W² is independently N or CR^{1′};
each R^{1′} is independently hydrogen, F, Cl, Br, I, CH₃, CH₂CH₃, Pr, i-Pr, n-Bu, i-Bu, t-Bu, CH₂CN, CH₂CO₂CH₃, CH₂C(═O)NH₂, CH₂C(═S)NH₂, C(═O)NH₂, C(═S)NH₂, C(═NH)NH₂, C(═O)NHOH, C(═O)NHNH₂, CH₂NH₃, NH₂, NHCH₃, N(CH₃)₂, NHCH₂CH₃, OH, OCH₃, OCH₂CH₃, SH, SCH₃, SCH₂CH₃, CO₂H, CN, or CHR*NH₂;
each R* is hydrogen, F, Cl, Br, or I;
each R^{2′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R^{3′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl;
each R⁵ is independently hydrogen, CH₃, CH₂CH₃, Pr, i-Pr, n-Bu, i-Bu, t-Bu, CH₂CN, CH₂CO₂CH₃, CH₂C(═O)NH₂, CH₂C(═S)NH₂, C(═O)NH₂, or C(═S)NH₂; and
such that there are no more than three ring-heteroatoms;
optionally in a pharmaceutically acceptable carrier or diluent.

38. The method of claim 37, wherein Z¹ is O.

39. The method of claim 37, wherein Z¹ is S.

40. The method of claim 37, wherein Z¹ is CH₂.

41. The method of claim 37, wherein Z¹ is CF₂.

42. A method for the treatment and/or prophylaxis of an HCV infection in a host in need thereof, comprising administering to said host an effective treatment amount of a β-D-nucleoside of the general formula (IV-b*): 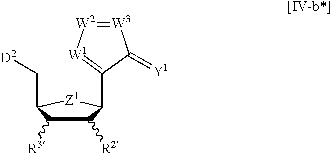
or a pharmaceutically acceptable salt and/or prodrug thereof, wherein:
each D² is independently OH, SH, NH₂, NHR⁴, or OD, wherein D is hydrogen, alkyl, acyl, monophosphate, diphosphate, triphosphate, monophosphate ester, diphosphate ester, triphosphate ester, phospholipid or amino acid;
each Z¹ is independently O, S, CH₂, CF₂, C(═O), or C(═CH₂);
each Y¹ is independently O, S, Se, or NH;
each W¹ and W² is independently N or CR^{1′};
each W³ is independently N, CH, CCH₃, CF, CCl, CBr, Cl, CCO₂H, CCO₂CH₃, CCONH₂, CC(═S)NH₂, or CCN;
each R^{1′} is independently hydrogen, halogen (F, Cl, Br or I), CH₃ (Me), CH₂CH₃ (Et), Pr, i-Pr, n-Bu, i-Bu, t-Bu, CH₂CN, CH₂CO₂CH₃, CH₂C(═O)NH₂, CH₂C(═S)NH₂, C(═O)NH₂, C(═S)NH₂, NH₂, NHCH₃, N(CH₃)₂, NHCH₂CH₃, OH, OCH₃, OCH₂CH₃, SH, SCH₃, SCH₂CH₃, CO₂H, or CN;
each R^{2′} independently is hydrogen, F, Cl, Br, 1, CH₃, CH₂OH, CH₂F, CH₂SH, CH₂SCH₃, CH₂N₃, CH₂NH₂, OH, OCH₃, or NH₂;
each R^{3′} independently is hydrogen, F, Cl, Br, 1, CH₃, CH₂OH, CH₂F, CH₂SH, CH₂SCH₃, CH₂N₃, CH₂NH₂, OH, OCH₃, or NH₂; and
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl;
optionally in a pharmaceutically acceptable carrier or diluent.

43. The method of claim 42, wherein Z¹ is O.

44. The method of claim 42, wherein Z¹ is S.

45. The method of claim 42, wherein Z¹ is CH₂.

46. The method of claim 42, wherein Z¹ is CF₂.

47. A method for the treatment and/or prophylaxis of an HCV infection in a host in need thereof, comprising administering to said host an effective treatment amount of a β-D-nucleoside of the general formula (IV-c*): 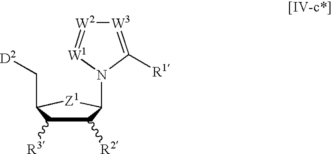
or a pharmaceutically acceptable salt and/or prodrug thereof, wherein:
each D² is independently OH, SH, NH₂, NHR⁴, or OD, wherein D is hydrogen, alkyl, acyl, monophosphate, diphosphate, triphosphate, monophosphate ester, diphosphate ester, triphosphate ester, phospholipid or amino acid;
each Z¹ is independently O, S, CH₂, CF₂, C(═O), or C(═CH₂);
each Y¹ is independently O, S, Se, or NH;
each W¹, W², and W³ is independently N or CR^{1′};
each R^{1′} is independently hydrogen, F, Cl, Br, I, CH₃, CH₂CH₃, Pr, i-Pr, n-Bu, i-Bu, t-Bu, CH₂CN, CH₂CO₂CH₃, CH₂C(═O)NH₂, CH₂C(═S)NH₂, C(═O)NH₂, C(═S)NH₂, NH₂, NHCH₃, N(CH₃)₂, NHCH₂CH₃, OH, OCH₃, OCH₂CH₃, SH, SCH₃, SCH₂CH₃, CO₂H, or CN;
each R^{2′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R^{3′} independently is hydrogen, F, Cl, Br, I, CH₃, CH₂OH, CH₂F, CH₂SH, CH₂SCH₃, CH₂N₃, CH₂NH₂, OH, OCH₃, or NH₂; and
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl;
optionally in a pharmaceutically acceptable carrier or diluent.

48. The method of claim 47, wherein Z¹ is O.

49. The method of claim 47, wherein Z¹ is S.

50. The method of claim 47, wherein Z¹ is CH₂.

51. The method of claim 47, wherein Z¹ is CF₂.

52. A method for the treatment and/or prophylaxis of an HCV infection in a host in need thereof, comprising administering to said host an effective treatment amount of a β-D-nucleoside of the general formula (IV-d*): 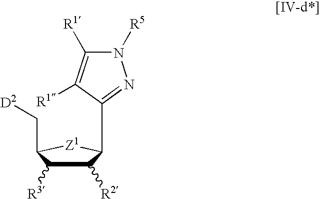
or a pharmaceutically acceptable salt and/or prodrug thereof, wherein:
each D² is independently OH, SH, NH₂, NHR⁴, or OD, wherein D is hydrogen, alkyl, acyl, monophosphate, diphosphate, triphosphate, monophosphate ester, diphosphate ester, triphosphate ester, phospholipid or amino acid;
each Z¹ is independently O, S, CH₂, CF₂, C(═O), or C(═CH₂);
each R^{1′} is independently CN, CO₂CH₃, C(═O)NH₂, C(═S)NH₂, or C(═NH)NH₂;
each R^{1″} is independently OH, SH, NH₂, or NHR⁵;
each R^{2′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R^{3′} independently is hydrogen, F, Cl, Br, I, CH₃, CH₂OH, CH₂F, CH₂SH, CH₂SCH₃, CH₂N₃, CH₂NH₂, OH, OCH₃, or NH₂;
each R⁴ is independently is hydrogen, optionally substituted or unsubstituted lower alkyl, lower haloalkyl, optionally substituted or unsubstituted lower alkenyl, lower haloalkenyl, optionally substituted or unsubstituted aryl, arylalkyl such as unsubstituted or substituted phenyl or benzyl, or an optionally substituted or unsubstituted acyl; and
each R⁵ is independently is hydrogen, optionally substituted or unsubstituted lower alkyl, or an optionally substituted or unsubstituted acyl;
optionally in a pharmaceutically acceptable carrier or diluent.

53. The method of claim 52, wherein Z¹ is O.

54. The method of claim 52, wherein Z¹ is S.

55. The method of claim 52, wherein Z¹ is CH₂.

56. The method of claim 52, wherein Z¹ is CF₂.

57. A method for the treatment and/or prophylaxis of an HCV infection in a host in need thereof, comprising administering to said host an effective treatment amount of a β-D-nucleoside of the formula: 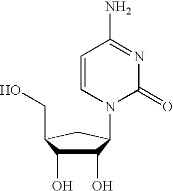
or a pharmaceutically acceptable salt and/or prodrug thereof, optionally in a pharmaceutically acceptable carrier or diluent.

58. A method for the treatment and/or prophylaxis of an HCV infection in a host in need thereof, comprising administering to said host an effective treatment amount of a β-D-nucleoside of the formula: 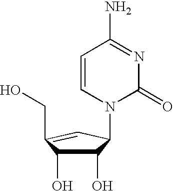
or a pharmaceutically acceptable salt and/or prodrug thereof, optionally in a pharmaceutically acceptable carrier or diluent.

59. A method for the treatment and/or prophylaxis of an HCV infection in a host in need thereof, comprising administering to said host an effective treatment amount of a β-D-nucleoside of the formula: 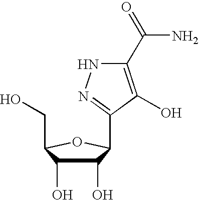
or a pharmaceutically acceptable salt and/or prodrug thereof, optionally in a pharmaceutically acceptable carrier or diluent.

60. A method for the treatment and/or prophylaxis of an HCV infection in a host in need thereof, comprising administering to said host an effective treatment amount of a β-D-nucleoside of the formula: 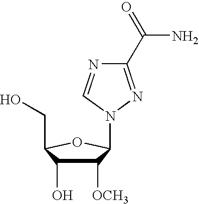
or a pharmaceutically acceptable salt and/or prodrug thereof, optionally in a pharmaceutically acceptable carrier or diluent.

61. A method for the treatment and/or prophylaxis of an HCV infection in a host in need thereof, comprising administering to said host an effective treatment amount of N-(phosphonoacetyl)-L-aspartate (PALA), or its pharmaceutically acceptable salt and/or prodrug, optionally in a pharmaceutically acceptable carrier or diluent.

62. The method of claim 37, wherein the pharmaceutically acceptable carrier is suitable for oral delivery.

63. The method of claim 37, wherein the pharmaceutically acceptable carrier is suitable for intravenous delivery.

64. The method of claim 37, wherein the pharmaceutically acceptable carrier is suitable for parenteral delivery.

65. The method of claim 37, wherein the pharmaceutically acceptable carrier is suitable for intradermal delivery.

66. The method of claim 37, wherein the pharmaceutically acceptable carrier is suitable for subcutaneous delivery.

67. The method of claim 37, wherein the pharmaceutically acceptable carrier is suitable for topical delivery.

68. The method of claim 37, wherein the effective compound is in the form of a dosage unit, such that said dosage unit contains 10 to 1500 mg of the compound.

69. The method of claim 37, wherein the effective compound is in the form of a dosage unit that is a tablet or capsule.

70. The method of claim 37, wherein the host is a human.
